(19) United States
(12) Patent Application Publication
(10) Pub. No.: US 2019/0100498 A1
(43) Pub. Date: Apr. 4, 2019

(54) CRYSTALLINE FORM

(71) Applicant: AstraZeneca AB, Södertälje (SE)

(72) Inventors: Thomas LANGER, Macclesfield (GB); Paul Allen BETHEL, Macclesfield (GB); Mohammed PERVEZ, Macclesfield (GB); Lai Chun CHAN, Macclesfield (GB); Sophie JANBON, Macclesfield (GB)

(21) Appl. No.: 16/086,750

(22) PCT Filed: Mar. 24, 2016
(86) PCT No.: PCT/EP2016/056664

§ 371 Date: Sep. 20, 2018

(51) Int. Cl. C07D 241/20; A61P 11/00

(52) U.S. Cl. CPC C07D 241/20; C07B 2200/13; A61P 11/00

(57) ABSTRACT

The application relates to a crystalline form of the compound of formula (I).

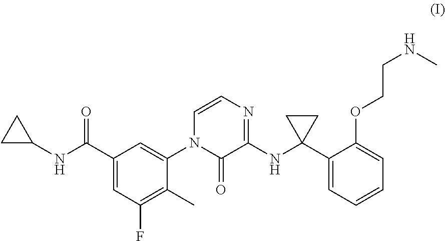 [SMILES: CNCCOc1ccccc1C1(Nc2nccn(-c3cc(C(=O)NC4CC4)cc(F)c3C)c2=O)CC1]

The present specification concerns a novel crystalline form of the compound of formula (I).

International Patent Application WO2009/001132, which is incorporated by reference, discloses pyrazinone derivatives useful for treating respiratory diseases, processes for their preparation and pharmaceutical compositions thereof. In particular, WO2009/001132 discloses, as experimental example 259 on page 228, the compound N-cyclopropyl-3-fluoro-4-methyl-5-[3-[[1-[2-[2-(methylamino)ethoxy]phenyl] cyclopropyl]amino]-2-oxo-1(2H)-pyrazinyl]-benzamide (hereafter referred to as the compound of formula (I)). The structure of the compound of formula (I) is shown below.

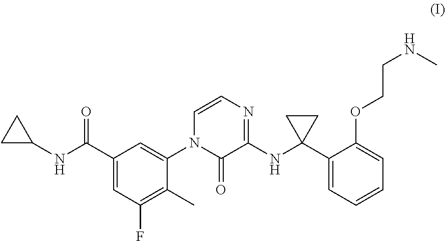 [SMILES: CNCCOc1ccccc1C1(Nc2nccn(-c3cc(C(=O)NC4CC4)cc(F)c3C)c2=O)CC1]

Crystallization is often the final step during the synthesis of an active pharmaceutical ingredient and the crystallization process can impact the physical attributes of the material such as its particle size, morphology, and polymorphic form. These properties may not only be important for effective downstream processing such as milling, granulation, tabletting and micronisation, but may also be necessary to satisfy product performance requirements. As the skilled person will be aware, typically it is not possible to predict, from molecular structure alone, what the crystallization behavior of a compound will be, nor what the physical properties of any given crystalline form will be.

The purification process described in WO2009/001132 uses reverse-phase high-pressure liquid chromatography (RPHPLC), and no separate crystallization process is described. The use of RPHPLC to isolate the compound of formula (I) may be unattractive for use at commercial scale. The crystallization process described in International Patent Application WO2010/071583, which is incorporated by reference, uses an ethyl acetate-heptane solvent system to obtain Form A of the compound of formula (I) (Form A). The crystalline particles of Form A produced using this method are relatively thin needles with high length-to-width aspect ratio (>20) and are electrostatic in nature. Such particles may be poorly suited for micronisation and may produce low micronized yields at commercial scale. Typically, micronisation losses of up to about 50% are observed due to blockage of the milling equipment using Form A as obtained by the process described in WO2010/071583.

We have now found a new crystalline form of the compound of formula (I) (Form S). Form S unexpectedly has a number of advantages over the previous Form A. For example, Form S exhibits improved chemical stability and therefore may result in a longer shelf-life for the pharmaceutical product. The crystalline particles of Form S also allow for improved flowability and reduced material adherence during the micronisation process. For example, for Form S the typical micronisation losses are less than 5%.

Accordingly, there is provided a crystalline form of N-cyclopropyl-3-fluoro-4-methyl-5-[3-[[1-[2-[2-(methylamino)ethoxy]phenyl] cyclopropyl]amino]-2-oxo-1(2H)-pyrazinyl]-benzamide, which has an X-ray powder diffraction pattern with at least one specific peak at about 2-theta=9.1°.

In a further aspect there is provided a crystalline form of N-cyclopropyl-3-fluoro-4-methyl-5-[3-[[1-[2-[2-(methylamino)ethoxy]phenyl] cyclopropyl]amino]-2-oxo-1(2H)-pyrazinyl]-benzamide, which has an X-ray powder diffraction pattern with at least one specific peak at about 2-theta=15.1°.

In a further aspect there is provided a crystalline form of N-cyclopropyl-3-fluoro-4-methyl-5-[3-[[1-[2-[2-(methylamino)ethoxy]phenyl] cyclopropyl]amino]-2-oxo-1(2H)-pyrazinyl]-benzamide, which has an X-ray powder diffraction pattern with at least one specific peak selected from the peaks at about 2-theta=9.1, 15.1, 16.2, 16.8 and 23.8°.

In a further aspect there is provided a crystalline form of N-cyclopropyl-3-fluoro-4-methyl-5-[3-[[1-[2-[2-(methylamino)ethoxy]phenyl] cyclopropyl]amino]-2-oxo-1(2H)-pyrazinyl]-benzamide, which has an X-ray powder diffraction pattern with at least two specific peaks at about 2-theta=9.1° and 15.1°.

In a further aspect there is provided a crystalline form of N-cyclopropyl-3-fluoro-4-methyl-5-[3-[[1-[2-[2-(methylamino)ethoxy]phenyl] cyclopropyl]amino]-2-oxo-1(2H)-pyrazinyl]-benzamide, which has an X-ray powder diffraction pattern with specific peaks at about 2-theta=9.1, 15.1, 16.2, 16.8 and 23.8°.

In a further aspect there is provided a crystalline form of N-cyclopropyl-3-fluoro-4-methyl-5-[3-[[1-[2-[2-(methylamino)ethoxy]phenyl] cyclopropyl]amino]-2-oxo-1(2H)-pyrazinyl]-benzamide, which has an X-ray powder diffraction pattern with specific peaks at about 2-theta=9.1, 11.6, 13.7, 15.1, 15.5, 16.2, 16.8, 18.1, 20.8 and 23.8°.

In a further aspect there is provided a crystalline form of N-cyclopropyl-3-fluoro-4-methyl-5-[3-[[1-[2-[2-(methylamino)ethoxy]phenyl] cyclopropyl]amino]-2-oxo-1(2H)-pyrazinyl]-benzamide, which has an X-ray powder diffraction pattern substantially the same as the X-ray powder diffraction pattern shown in FIG. 1.

In a further aspect there is provided a crystalline form of N-cyclopropyl-3-fluoro-4-methyl-5-[3-[[1-[2-[2-(methylamino)ethoxy]phenyl] cyclopropyl]amino]-2-oxo-1(2H)-pyrazinyl]-benzamide, which has an X-ray powder diffraction pattern with at least one specific peak at 2-theta=9.1° plus or minus 0.2° 2-theta.

In a further aspect there is provided a crystalline form of N-cyclopropyl-3-fluoro-4-methyl-5-[3-[[1-[2-[2-(methylamino)ethoxy]phenyl] cyclopropyl]amino]-2-oxo-1(2H)-pyrazinyl]-benzamide, which has an X-ray powder diffraction pattern with at least one specific peak at 2-theta=15.1° plus or minus 0.2° 2-theta.

In a further aspect there is provided a crystalline form of N-cyclopropyl-3-fluoro-4-methyl-5-[3-[[1-[2-[2-(methylamino)ethoxy]phenyl] cyclopropyl]amino]-2-oxo-1(2H)-pyrazinyl]-benzamide, which has an X-ray powder diffraction pattern with at least one specific peak selected from the peaks at 2-theta=9.1, 15.1, 16.2, 16.8 and 23.8° wherein said values may be plus or minus 0.2° 2-theta.

In a further aspect there is provided a crystalline form of N-cyclopropyl-3-fluoro-4-methyl-5-[3-[[1-[2-[2-(methylamino)ethoxy]phenyl] cyclopropyl]amino]-2-oxo-1(2H)-pyrazinyl]-benzamide, which has an X-ray powder diffraction pattern with at least two specific peaks at 2-theta=9.1° and 15.1° wherein said values may be plus or minus 0.2° 2-theta.

In a further aspect there is provided a crystalline form of N-cyclopropyl-3-fluoro-4-methyl-5-[3-[[1-[2-[2-(methylamino)ethoxy]phenyl] cyclopropyl]amino]-2-oxo-1(2H)-pyrazinyl]-benzamide, which has an X-ray powder diffraction pattern with specific peaks at 2-theta=9.1, 15.1, 16.2, 16.8 and 23.8° wherein said values may be plus or minus 0.2° 2-theta.

In a further aspect there is provided a crystalline form of N-cyclopropyl-3-fluoro-4-methyl-5-[3-[[1-[2-[2-(methylamino)ethoxy]phenyl] cyclopropyl]amino]-2-oxo-1(2H)-pyrazinyl]-benzamide, which has an X-ray powder diffraction pattern with specific peaks at 2-theta=9.1, 11.6, 13.7, 15.1, 15.5, 16.2, 16.8, 18.1, 20.8 and 23.8° wherein said values may be plus or minus 0.2° 2-theta.

In a further aspect there is provided a crystalline form of N-cyclopropyl-3-fluoro-4-methyl-5-[3-[[1-[2-[2-(methylamino)ethoxy]phenyl] cyclopropyl]amino]-2-oxo-1(2H)-pyrazinyl]-benzamide, which has an X-ray powder diffraction pattern with at least one specific peak at 2-theta=9.1° plus or minus 0.1° 2-theta.

In a further aspect there is provided a crystalline form of N-cyclopropyl-3-fluoro-4-methyl-5-[3-[[1-[2-[2-(methylamino)ethoxy]phenyl] cyclopropyl]amino]-2-oxo-1(2H)-pyrazinyl]-benzamide, which has an X-ray powder diffraction pattern with at least one specific peak at 2-theta=15.1° plus or minus 0.1° 2-theta.

In a further aspect there is provided a crystalline form of N-cyclopropyl-3-fluoro-4-methyl-5-[3-[[1-[2-[2-(methylamino)ethoxy]phenyl] cyclopropyl]amino]-2-oxo-1(2H)-pyrazinyl]-benzamide, which has an X-ray powder diffraction pattern with at least one specific peak selected from the peaks at 2-theta=9.1, 15.1, 16.2, 16.8 and 23.8° wherein said values may be plus or minus 0.1° 2-theta.

In a further aspect there is provided a crystalline form of N-cyclopropyl-3-fluoro-4-methyl-5-[3-[[1-[2-[2-(methylamino)ethoxy]phenyl] cyclopropyl]amino]-2-oxo-1(2H)-pyrazinyl]-benzamide, which has an X-ray powder diffraction pattern with at least two specific peaks at 2-theta=9.1° and 15.1° wherein said values may be plus or minus 0.1° 2-theta.

In a further aspect there is provided a crystalline form of N-cyclopropyl-3-fluoro-4-methyl-5-[3-[[1-[2-[2-(methylamino)ethoxy]phenyl] cyclopropyl]amino]-2-oxo-1(2H)-pyrazinyl]-benzamide, which has an X-ray powder diffraction pattern with specific peaks at 2-theta=9.1, 15.1, 16.2, 16.8 and 23.8° wherein said values may be plus or minus 0.1° 2-theta.

In a further aspect there is provided a crystalline form of N-cyclopropyl-3-fluoro-4-methyl-5-[3-[[1-[2-[2-(methylamino)ethoxy]phenyl] cyclopropyl]amino]-2-oxo-1(2H)-pyrazinyl]-benzamide, which has an X-ray powder diffraction pattern with specific peaks at 2-theta=9.1, 11.6, 13.7, 15.1, 15.5, 16.2, 16.8, 18.1, 20.8 and 23.8° wherein said values may be plus or minus 0.1° 2-theta.

When it is stated herein that the present specification relates to a crystalline form of a compound disclosed herein, such as the compound of formula (I), the degree of crystallinity is conveniently greater than about 60%, more conveniently greater than about 80%, preferably greater than about 90% and more preferably greater than about 95%. Most preferably the degree of crystallinity is greater than about 98%.

Form S of N-cyclopropyl-3-fluoro-4-methyl-5-[3-[[1-[2-[2-(methylamino)ethoxy]phenyl] cyclopropyl]amino]-2-oxo-1(2H)-pyrazinyl]-benzamide provides X-ray powder diffraction patterns substantially the same as the X-ray powder diffraction pattern shown in FIG. 1 and exhibits the ten (angle 2-theta values) shown in Table A. It will be understood that the 2-theta values of the X-ray powder diffraction pattern may vary slightly from one machine to another or from one sample to another, and so the values quoted are not to be construed as absolute.

It is known that an X-ray powder diffraction pattern may be obtained which has one or more measurement errors depending on measurement conditions (such as equipment, sample preparation or machine used). In particular, it is generally known that intensities in an X-ray powder diffraction pattern may fluctuate depending on measurement conditions. Therefore it should be understood that the crystalline form (Form S) of N-cyclopropyl-3-fluoro-4-methyl-5-[3-[[1-[2-[2-(methylamino)ethoxy]phenyl] cyclopropyl]amino]-2-oxo-1(2H)-pyrazinyl]-benzamide is not limited to crystals that provide X-ray powder diffraction patterns identical to the X-ray powder diffraction pattern shown in FIG. 1, and any crystals providing X-ray powder diffraction patterns substantially the same as those shown in FIG. 1 fall within the scope of the present invention. A person skilled in the art of X-ray powder diffraction is able to judge the substantial identity of X-ray powder diffraction patterns.

Persons skilled in the art of X-ray powder diffraction will understand that the relative intensity of peaks can be affected by, for example, grains above 30 microns in size and non-unitary aspect ratios, which may affect analysis of samples. The skilled person will also understand that the position of reflections can be affected by the precise height at which the sample sits in the diffractometer and the zero calibration of the diffractometer. The surface planarity of the sample may also have a small effect. Hence the diffraction pattern data presented are not to be taken as absolute values (for further information see Jenkins, R & Snyder, R. L. ‘Introduction to X-Ray Powder Diffractometry’ John Wiley & Sons 1996; Bunn, C. W. (1948), Chemical Crystallography, Clarendon Press, London; Klug, H. P. & Alexander, L. E. (1974), X-Ray Diffraction Procedures).

Generally, a measurement error of a diffraction angle in an X-ray powder diffractogram is approximately plus or minus 0.2° 2-theta, and such degree of a measurement error should be taken into account when considering the X-ray powder diffraction pattern in FIG. 1 and when reading Table A. Furthermore, it should be understood that intensities might fluctuate depending on experimental conditions and sample preparation (preferred orientation).

In a further aspect there is provided a crystalline form of N-cyclopropyl-3-fluoro-4-methyl-5-[3-[[1-[2-[2-(methylamino)ethoxy]phenyl] cyclopropyl]amino]-2-oxo-1(2H)-pyrazinyl]-benzamide, which has an FT-Raman spectrum substantially the same as the FT-Raman spectrum shown in FIG. 2.

Because of the improved chemical stability and potentially longer shelf-life of Form S compared to Form A, Form S may be particularly suited to formulation as a pharmaceutical composition.

The pharmaceutical compositions of this specification may be administered in standard manner for the disease condition that it is desired to treat, for example by topical (such as to the lung and/or airways or to the skin), oral, rectal or parenteral administration. For these purposes the compound of formula (I) may be formulated by means known in the art into the form of, for example, aerosols, dry powder formulations, tablets, capsules, syrups, powders, granules, aqueous or oily solutions or suspensions, (lipid) emulsions, dispersible powders, suppositories, ointments, creams, drops and sterile injectable aqueous or oily solutions or suspensions.

A particular pharmaceutical composition of this specification is one suitable for inhaled administration, inhalation being a particularly useful method for administering the compound of formula (I) when treating respiratory diseases such as chronic obstructive pulmonary disease (COPD) or asthma.

When administered by inhalation, metered dose inhaler devices may be used to administer the active ingredient, dispersed in a suitable propellant and with or without additional excipients such as ethanol, surfactants, lubricants or stabilising agents. Suitable propellants include hydrocarbon, chlorofluorocarbon and hydrofluoroalkane (e.g. heptafluoroalkane) propellants, or mixtures of any such propellants. Preferred propellants are P134a and P227, each of which may be used alone or in combination with other propellants and/or surfactant and/or other excipients. Nebulised aqueous suspensions or, preferably, solutions may also be employed, with or without a suitable pH and/or tonicity adjustment, either as a unit-dose or multi-dose formulations.

Dry powder inhalers may be used to administer the active ingredient, alone or in combination with a pharmaceutically acceptable carrier, such as lactose, in the later case either as a finely divided powder or as an ordered mixture. The dry powder inhaler may be single dose or multi-dose and may utilise a dry powder or a powder-containing capsule. Metered dose inhaler, nebuliser and dry powder inhaler devices are well known and a variety of such devices are available.

Accordingly, in a further aspect of the specification there is provided a pharmaceutical composition that comprises a crystalline form of N-cyclopropyl-3-fluoro-4-methyl-5-[3-[[1-[2-[2-(methylamino)ethoxy]phenyl]cyclopropyl]amino]-2-oxo-1(2H)-pyrazinyl]-benzamide, as described herein (Form S), and a pharmaceutically acceptable diluent or carrier.

In another embodiment, there is provided a pharmaceutical composition that comprises a crystalline form of N-cyclopropyl-3-fluoro-4-methyl-5-[3-[[1-[2-[2-(methylamino)ethoxy]phenyl]cyclopropyl]amino]-2-oxo-1(2H)-pyrazinyl]-benzamide, as described herein (Form S), and a pharmaceutically acceptable diluent or carrier, which is formulated for inhaled administration.

In another embodiment, there is provided a pharmaceutical composition that comprises a crystalline form of N-cyclopropyl-3-fluoro-4-methyl-5-[3-[[1-[2-[2-(methylamino)ethoxy]phenyl]cyclopropyl]amino]-2-oxo-1(2H)-pyrazinyl]-benzamide, as described herein (Form S), and lactose, which is formulated for inhaled administration.

The biological activity of the compound of formula (I) has been measured in WO2009/001132 at pIC50=10.0 in a p38 enzyme inhibition assay. Hence the compound of formula (I) has activity as a p38 kinase inhibitor. The compound of formula (I) is therefore suitable for the treatment of inflammatory diseases such as (but not restricted to) rheumatoid arthritis, osteoarthritis, asthma, allergic rhinitis, chronic obstructive pulmonary disease (COPD), psoriasis and inflammatory bowel disease. In particular the compound of formula (I) is useful for the treatment of respiratory diseases, such as asthma and COPD.

Accordingly, in a further aspect there is provided a crystalline form of N-cyclopropyl-3-fluoro-4-methyl-5-[3-[[1-[2-[2-(methylamino)ethoxy]phenyl]cyclopropyl]amino]-2-oxo-1(2H)-pyrazinyl]-benzamide, as described herein (Form S), for use in therapy.

In a further aspect there is provided a crystalline form of N-cyclopropyl-3-fluoro-4-methyl-5-[3-[[1-[2-[2-(methylamino)ethoxy]phenyl] cyclopropyl]amino]-2-oxo-1(2H)-pyrazinyl]-benzamide, as described herein (Form S), for use in the treatment of respiratory diseases, such as asthma and COPD.

In a yet further aspect there is provided a crystalline form of N-cyclopropyl-3-fluoro-4-methyl-5-[3-[[1-[2-[2-(methylamino)ethoxy]phenyl] cyclopropyl]amino]-2-oxo-1(2H)-pyrazinyl]-benzamide, as described herein (Form S), for use in the treatment of COPD.

In a still further aspect there is provided the use of a crystalline form of N-cyclopropyl-3-fluoro-4-methyl-5-[3-[[1-[2-[2-(methylamino)ethoxy]phenyl] cyclopropyl]amino]-2-oxo-1(2H)-pyrazinyl]-benzamide, as described herein (Form S), in the manufacture of a medicament for use in the treatment of COPD.

In a yet further aspect there is provided a method of treating COPD in a warm-blooded animal, such as man, which comprises administering to a mammal in need of such treatment a therapeutically effective amount of a crystalline form of N-cyclopropyl-3-fluoro-4-methyl-5-[3-[[1-[2-[2-(methylamino)ethoxy]phenyl] cyclopropyl]amino]-2-oxo-1(2H)-pyrazinyl]-benzamide, as described herein (Form S),

In WO2010/071583 the compound of formula (I) is found to also be suitable for use in the treatment of respiratory diseases when in combination with various second active ingredients.

Accordingly, in a further aspect of the specification there is provided a pharmaceutical product comprising, in combination, a first active ingredient which is the crystalline form of N-cyclopropyl-3-fluoro-4-methyl-5-[3-[[1-[2-[2-(methylamino)ethoxy]phenyl] cyclopropyl]amino]-2-oxo-1(2H)-pyrazinyl]-benzamide, as described herein (Form S), and a second active ingredient selected from:

a muscarinic antagonist;

a β₂ adrenoceptor agonist;

a dual β₂ adrenoceptor agonist/M₃ receptor anatagonist (MABA compound);

a steroidal glucocorticoid receptor agonist;

a non-steroidal glucocorticoid receptor agonist;

an IKK2 kinase inhibitor;

a phosphodiesterase PDE4 inhibitor; or

an inhibitor of neutrophil elastase.

In a further aspect there is provided a pharmaceutical product comprising, in combination, a first active ingredient which is the crystalline form of N-cyclopropyl-3-fluoro-4-methyl-5-[3-[[1-[2-[2-(methylamino)ethoxy]phenyl] cyclopropyl]amino]-2-oxo-1(2H)-pyrazinyl]-benzamide, as described herein (Form S), and a second active ingredient which is a steroidal glucocorticoid receptor agonist.

In a further aspect there is provided a pharmaceutical product comprising, in combination, a first active ingredient which is the crystalline form of N-cyclopropyl-3-fluoro-4-methyl-5-[3-[[1-[2-[2-(methylamino)ethoxy]phenyl] cyclopropyl]amino]-2-oxo-1(2H)-pyrazinyl]-benzamide, as described herein (Form S), and a second active ingredient which is budesonide.

In a further aspect there is provided a pharmaceutical product comprising, in combination, a first active ingredient which is the crystalline form of N-cyclopropyl-3-fluoro-4-methyl-5-[3-[[1-[2-[2-(methylamino)ethoxy]phenyl] cyclopropyl]amino]-2-oxo-1(2H)-pyrazinyl]-benzamide, as described herein (Form S), and a second active ingredient which is a non-steroidal glucocorticoid receptor agonist.

In a yet further aspect there is provided a pharmaceutical product comprising, in combination, a first active ingredient which is the crystalline form of N-cyclopropyl-3-fluoro-4-methyl-5-[3-[[1-[2-[2-(methylamino)ethoxy]phenyl] cyclopropyl]amino]-2-oxo-1(2H)-pyrazinyl]-benzamide, as described herein (Form S), and a second active ingredient which is 3-{5-[(1R,2S)-2-[(2,2-difluoropropanoyl)amino]-1-(2,3-dihydro-1,4-benzodioxin-6-yl)propoxy]-1H-indazol-1-yl}-N-[(3R)-tetrahydro-3-furanyl]benzamide [WO2009/142571, Example 6].

In another aspect there is provided a new crystallization process for the compound of formula (I). In another aspect there is provided a crystallization process which comprises crystallizing the compound of formula (I) from a solution, suspension or slurry of the compound of formula (I) in a solvent system containing acetonitrile. In a further aspect, the crystallization process comprises crystallizing the compound of formula (I) from a solution, suspension or slurry of the compound of formula (I) in acetonitrile. In a further aspect the crystallization process is a cooling crystallization process, wherein the solution, suspension or slurry of the compound of formula (I) in acetonitrile is heated in order to solubilise the contents before cooling to a desired isolation temperature. In another aspect, the solution, suspension or slurry is heated to between 60 to 90° C., such as 75 to 85° C., such as about 80° C. In a further aspect the heated solution is cooled to an isolation temperature of between 0 to 15° C., such as about 5° C. In a further aspect, the cooling rate of the solution is between 0.01 to 0.05° C./min, such as about 0.04° C./min. In a further aspect, the crystallization process may comprise the addition of seed crystals of the compound of formula (I).

The general synthetic route to the compound of formula (I) starting from the compound of formula (II) is set out below in Scheme 1, wherein PG is an amine protecting group, and X is halogen. The synthesis of intermediate (II) is disclosed in WO2010/071583 as Preparation 4b.

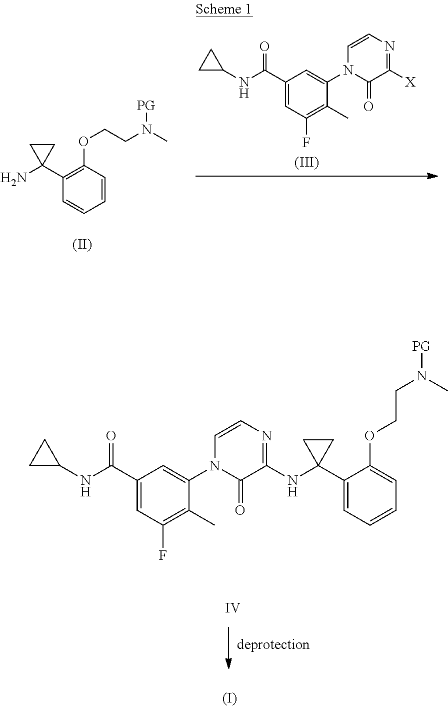

In one aspect, there is disclosed a new route to synthesise the pyrazinone intermediate of general formula (III), as described in Scheme 2 below, wherein X is halogen and R² is C₁₋₃ alkyl.

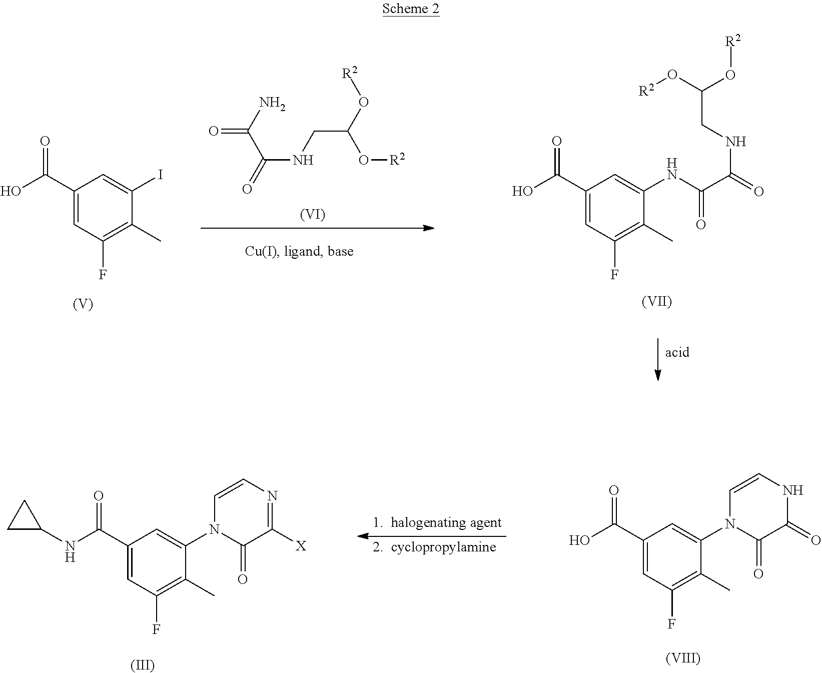

As used herein, a halogen atom is typically fluorine, chlorine, bromine or iodine.

As used herein, an alkyl group may be straight or branched chain, e.g. C₁₋₆ alkyl or C₁₋₄ alkyl. Examples of alkyl groups are methyl, ethyl, n-propyl, i-propyl, n-butyl, i-butyl, s-butyl, t-butyl, n-heptyl or n-hexyl, for example methyl, ethyl, i-propyl or t-butyl.

As used herein, a protecting group is a chemical moiety which is introduced into a molecule by reaction with a functional group in the said molecule in order to obtain chemoselectivity in a subsequent chemical reaction, and is thereafter removed in a later chemical reaction Amine protecting groups are well known to those skilled in the art, and are described, for example, in W. Greene and P. G. M. Wuts, Greene's Protective Groups in Organic Synthesis, Wiley-Interscience, 2006 or in P. J. Kocienski, Protecting Groups, Thieme, 2005. Examples of amine protecting groups include carbamates, acetamides, phthalimides, benzylamines, napthylamines, allylamines, tritylamines, imines and sulfonamides. The skilled person will be aware that the naming of such general classes of amine protecting groups may include reference to the amine nitrogen in the molecule to be protected. In the present specification, therefore, the skilled person will be aware that reference to PG may be taken to be a reference to the general class of amine protecting group (in which case the amine nitrogen will be included in the name, for example benzylamine), or may be taken to be a reference to the specific chemical moiety which is attached to the said amine nitrogen (in which case the amine nitrogen will not be included in the name, for example a benzyl group). Thus, by way of illustration only, where the class of amine protecting group is benzylamine, PG in the present application may be referred to as benzylamine (the general class) or as a benzyl group (the specific chemical moiety).

In a further aspect, therefore, there is provided a process for preparing a compound of formula (III), wherein X is halogen, comprising reacting a compound of formula (VIII) with (i) a halogenating agent and (ii) cyclopropylamine, and optionally thereafter forming a salt thereof.

As used herein, a halogenating agent is a reagent intended to transfer one or more halogen atoms into the molecule undergoing reaction. Examples of halogenating agents include, but are not limited to, phosphorus oxychloride, phosphorus oxybromide, thionyl chloride, thionyl bromide, N-bromosuccinimide and 1,3-Dibromo-5,5-dimethylhydantoin. In one aspect the halogenating agent is phosphorus oxybromide.

The halogenation reaction can be carried out in the presence of a base, such as an organic base, for example triethylamine or diisopropylethylamine. In one aspect, the base comprises triethylamine.

The reaction may be carried out in a variety of organic solvents. In one aspect the solvent comprises acetonitrile.

The reaction may be carried out at a variety of temperatures, for example at 75 to 80° C.

The compound of formula (VIII) can be obtained via an acid-catalysed cyclisation of a compound of formula (VII) (Scheme 3).

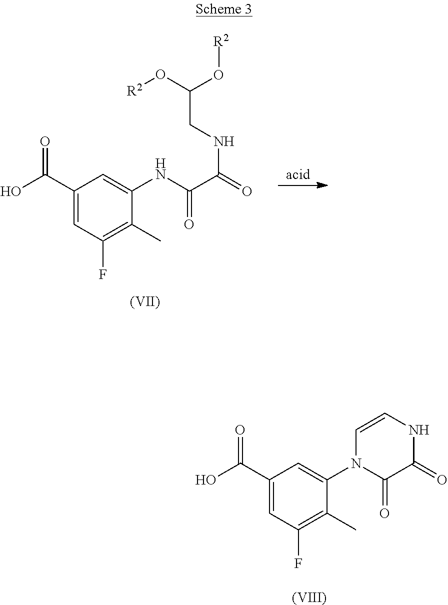

In another aspect, therefore, there is provided a process for preparing a compound of formula (VIII), comprising reacting a compound of formula (VII), wherein R² is C₁₋₃ alkyl, with acid, and optionally thereafter forming a salt thereof. In a further aspect R² is methyl.

The cyclisation reaction is carried out in the presence of acid. Suitable acids include organic acids for examples methanesulfonic acid, acetic acid or toluenesulfonic acid. In one aspect the acid comprises methanesulfonic acid.

The reaction may be carried out in a variety of solvents. In one aspect the solvent comprises acetic acid.

The reaction may be carried out at a variety of temperatures, for example at 105 to 110° C.

The compound of formula (VII) can be obtained via reaction of a compound of formula (V) with a compound of formula (VI) (Scheme 4).

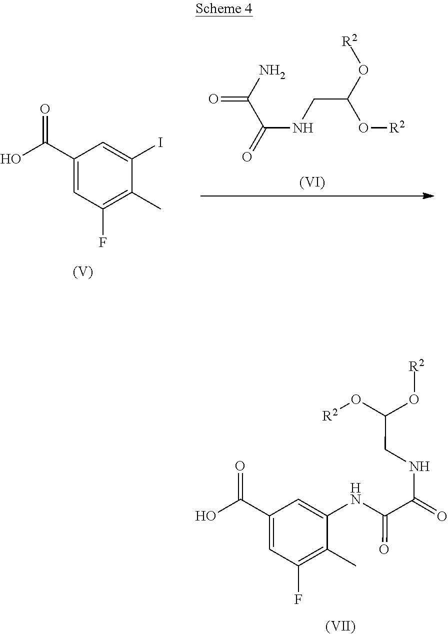

In another aspect, there is provided a process for preparing a compound of formula (VII), comprising reacting a compound of formula (V) with a compound of formula (VI), wherein R² is C₁₋₃ alkyl, and optionally thereafter forming a salt thereof. In a further aspect R² is methyl.

The reaction may be carried out under metal catalysis, such as copper catalysis, by methods known to those skilled in the art. For example, the reaction may be carried out utilizing a copper(I) catalyst, such as copper(I) iodide, a suitable ligand, such as trans-(1R,2R)—N,N′-bismethyl-1,2-cyclohexanediamine, and a suitable base, such as potassium carbonate.

The reaction may be carried out in a variety of organic solvents. In one aspect the solvent comprises dimethyl formamide (DMF).

The reaction may be carried out at a variety of temperatures, for example at 105 to 110° C.

In a further aspect, there is provided the compound of formula (VII), or a salt thereof, wherein R² is C₁₋₃ alkyl.

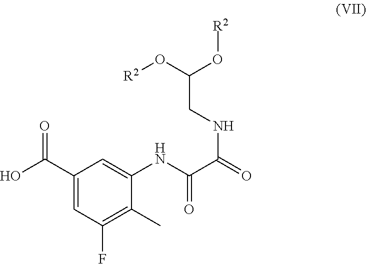

In a further aspect there is provided the compound 3-[[2-(2,2-dimethoxyethylamino)-2-oxo-acetyl]amino]-5-fluoro-4-methyl-benzoic acid, or a salt thereof. In a still further aspect there is provide the compound 3-[[2-(2,2-dimethoxyethylamino)-2-oxo-acetyl]amino]-5-fluoro-4-methyl-benzoic acid.

In a further aspect, there is provided the compound of formula (VIII), or a salt thereof.

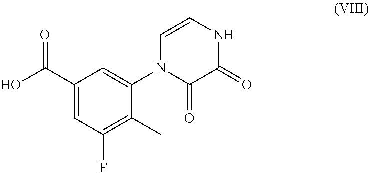 [SMILES: Cc1c(F)cc(C(=O)O)cc1-n1cc[nH]c(=O)c1=O]

In a still further aspect there is provided the compound 3-(2,3-dioxo-1H-pyrazin-4-yl)-5-fluoro-4-methyl-benzoic acid.

In a further aspect, there is provided the compound of formula (III), or a salt thereof, wherein X is halogen.

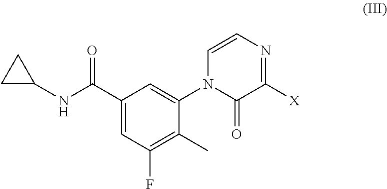

In a further aspect there is provided the compound 3-(3-bromo-2-oxo-pyrazin-1-yl)-N-cyclopropyl-5-fluoro-4-methyl-benzamide, or a salt thereof. In a still further aspect there is provided the compound 3-(3-bromo-2-oxo-pyrazin-1-yl)-N-cyclopropyl-5-fluoro-4-methyl-benzamide.

The various aspects of the specification are illustrated by the following Examples. These Examples are given by way of illustration only and are non-limiting.

### SUMMARY OF FIGURES

FIG. 1 shows the XRPD-diffractogram of crystalline Form S of the compound of formula (I).

FIG. 2 shows the FT-Raman spectrum of crystalline Form S of the compound of formula (I).

### ABBREVIATIONS

Ti(OⁱPr)₄ titanium tetraisopropoxide
BF₃.(Et₂O)₂ boron trifluoride etherate
EtMgBr ethyl magnesium bromide
Et₂Zn diethyl zinc
MeLi methyl lithium
ClTi(OⁱPr)₃ chlorotitanium triisopropoxide
DIPEA diisopropylethylamine
ⁱPrMgCl isopropyl magnesium chloride
Cs₂CO₃ cesium carbonate
Pd/C palladium on carbon
Pd₂(dba)₃ tris(dibenzylideneacetone)dipalladium(0)
Dppf 1,1′-bis(diphenylphosphino)ferrocene
NH₄⁺HCO₂_{^{−}} ammonium formate
NaH sodium hydride
TFA trifluoroacetic acid
LiI lithium iodide
LiOⁱPr lithium isopropoxide
DPPA diphenylphosphoryl azide
DMCDA trans-(1R,2R)—N,N′-bismethyl-1,2-cyclohexanediamine
PIFA bis[(trifluoroacetoxy)iodo]benzene
pTSA para-toluenesulfonic acid
TEA triethylamine
DCM dichloromethane
DMF N,N-dimethyl formamide
DMSO dimethyl sulfoxide
THF tetrahydrofuran
NaOH sodium hydroxide
MeOH methanol
EtOH ethanol
NaOMe sodium methoxide
2-MeTHF 2-methyltetrahydrofuran
IPA isopropyl alcohol
MeCN acetonitrile
MTBE methyl tert-butyl ether
H₂O₂ hydrogen peroxide
K₂CO₃ potassium carbonate
CbzCl benzyl chloroformate
ⁱPrOAc isopropyl acetate
^{t}BuOH tert-butanol
Et₃BnNCl benzyltriethylammonium chloride

### General Methods

Unless stated otherwise, starting materials were commercially available. All solvents and commercial reagents were of laboratory grade and were used as received. Unless stated otherwise, all operations were carried out at ambient temperature, i.e. in the range 17 to 28° C. and, where appropriate, under an atmosphere of an inert gas such as nitrogen.

Large scale reactions were carried out in stainless steel or glass-lined steel reactors fitted with heat transfer jackets and serviced with appropriate ancillary equipment.

When given, 1H NMR spectra were recorded on a Bruker Avance 600 (600 MHz), a Bruker DRX 500 (500 MHz), a Bruker 300 (300 MHz) or a Varian Unitylnova 500 MHz, 400 MHz or 300 MHz instrument. Either the central peaks of chloroform-d (CDCl3; δH 7.27 ppm), dimethylsulfoxide-d6 (d6-DMSO; δH 2.50 ppm) or methanol-d4 (CD3OD; δH 3.31 ppm), or an internal standard of tetramethylsilane (TMS; δH 0.00 ppm) were used as references. Sample solutions may also contain an internal standard (for example maleic acid, 2,3,5,6-tetrachloronitrobenzene or benzyl benzoate) for assay determination and/or added trifluoroacetic acid, to move exchangeable proton signals (e.g. from maleic acid) away from analyte resonances. Spectral data is reported as a list of chemical shifts (δ, in ppm) with a description of each signal, using standard abbreviations (s=singlet, d=doublet, m=multiplet, t=triplet, q=quartet, br=broad, etc.). It is well known in the art that chemical shifts and J-coupling constants may vary slightly as a result of sample preparation differences, for example analyte concentration and whether or not additives (for example NMR assay standards or trifluoroacetic acid) are included.

Mass spectra were recorded on an Agilent MSD (+ve and −ve APCI and/or electrospray (e.g. in multimode)) or a Waters Micromass ZQ (+ve and −ve electrospray) following analytical HPLC. Where values for m/z are given, generally only ions which indicate the parent mass are reported, and the mass ions quoted are the positive or negative mass ions: [M]+, [M+H]+, [M−H]− or [M+2H—BOC]+.

X-Ray powder diffraction analysis (XRPD) was performed on samples prepared according to standard methods, for example those described in Kitaigorodsky, A. I. (1973), Molecular Crystals and Molecules, Academic Press, New York; Giacovazzo, C. et al (1995), Fundamentals of Crystallography, Oxford University Press; Jenkins, R. and Snyder, R. L. (1996), Introduction to X-Ray Powder Diffractometry, John Wiley & Sons, New York; Bunn, C. W. (1948), Chemical Crystallography, Clarendon Press, London; or Klug, H. P. & Alexander, L. E. (1974), X-ray Diffraction Procedures, John Wiley and Sons, New York. X-ray powder diffraction data were measured with and without Corundum as an internal reference. The X-ray powder diffraction pattern was determined by mounting a sample on a zero background holder, single silicon crystal, and spreading out the sample into a thin layer.

X-ray analyses were performed using a Theta-Theta PanAlytical X'Pert Pro instrument using a copper anode with a nickel filter (X-ray Kal wavelength=1.5418 Å) at 45 KV and 40 mA. Automatic variable divergence and anitscatter slits were used and the samples were rotated during measurement. Samples were scanned from 2.4-50° 2-Theta using a 0.013° step width and a 115.77 s count time together with a PIXCEL detector (active length 3.35° 2-Theta). The XRPD patterns were obtained in Bragg-Brentano geometry.

It is known in the art that an X-ray powder diffraction pattern may be obtained which has one or more measurement errors depending on measurement conditions (such as equipment, sample preparation or machine used). In particular, it is generally known that intensities in an X-ray powder diffraction pattern may fluctuate depending on measurement conditions and sample preparation. For example, persons skilled in the art of X-ray powder diffraction will realise that the relative intensities of the peaks may vary according to the orientation of the sample under test and on the type and setting of the instrument used. The skilled person will also realise that the position of reflections can be affected by the precise height at which the sample sits in the diffractometer and the zero calibration of the diffractometer. The surface planarity of the sample may also have a small effect. Hence a person skilled in the art will appreciate that the diffraction pattern data presented herein is not to be construed as absolute and any crystalline form that provides a powder diffraction pattern substantially identical to those disclosed herein fall within the scope of the present disclosure. Generally, a measurement error of a diffraction angle in an X-ray powder diffraction pattern is about 5% or less, typically plus or minus 0.2° 2-theta.

FT-Raman spectra were measured using a Bruker FT-Raman MultiRAM instrument according to standard procedures, equipped with a Nd:YAG (1064 nm) laser and an LN—Ge diode detector. The laser power was set to 1000 mW and the resolution to 2 cm⁻¹. The accuracy of the wavelength calibration was ±1 cm-1. The laser beam was defocused and an aperture of 5 mm was used. The samples, approximately 30 mg, were placed in a 96-well plate and the instrument HTS mode was used to collect the spectra. The wavelength shifts of specific peaks between different crystalline forms are small, hence account must be taken of the interrelationship of several peaks and the overall spectral signal to distinguish between forms. The spectra were not corrected for instrument response.

### Example 1: Preparation of 1-[2-[2-[benzyl(methyl)amino)ethoxy]phenyl]cyclopropan-amine di para-toluenesulfonic acid (Scheme 5)

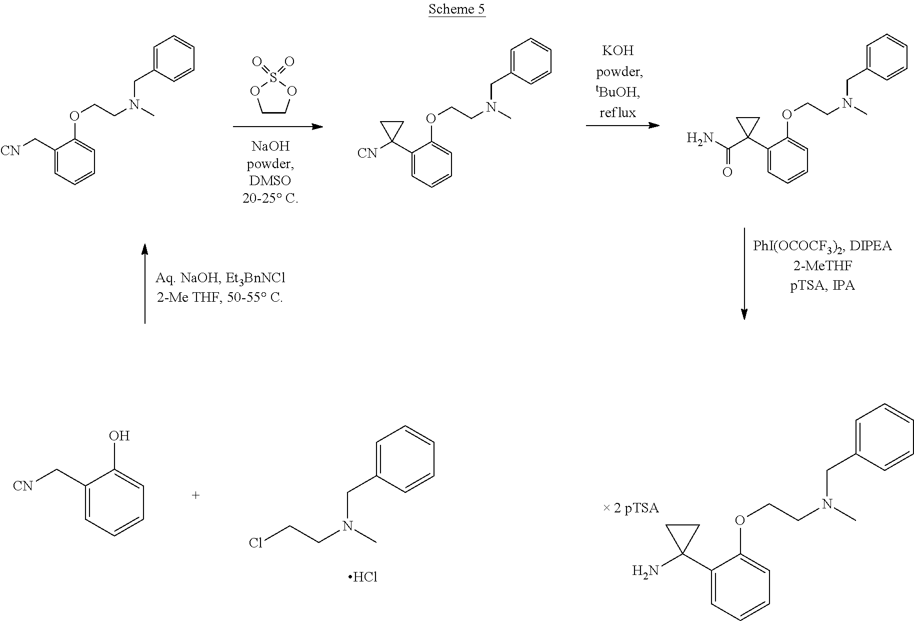

### Step 1: 2-[2-[2-[benzyl(methyl)amino]ethoxy]phenyl]acetonitrile

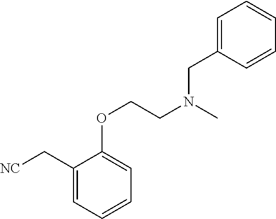 [SMILES: CN(CCOc1ccccc1CC#N)Cc1ccccc1]

To a mixture of 2-(2-hydroxyphenyl)acetonitrile (100 g, 0.74 moles, 1.0 eq), N-benzyl-2-chloro-N-methyl-ethanamine hydrochloride (163.53 g, 0.72 moles, 0.98 eq) and benzyltriethylammonium chloride (34.25 g, 0.15 moles, 0.2 eq) in 2-methyltetrahydrofuran (1000 ml, 10 rel vol), aqueous sodium hydroxide (20% w/v, 248.47 ml, 2.0 eq) was added and the reaction mixture was heated to 50-55° C. under stirring. After 4 h at 50-55° C., another lot of aqueous sodium hydroxide (20% w/v, 62.12 ml, 0.5 eq) was added and agitation was continued at 50-55° C. for further 16 h. Additional aqueous sodium hydroxide (20% w/v, 62.12 ml, 0.5 eq) was added and the mixture was stirred for another 8 h. The reaction was monitored by HPLC for completion (2-(2-hydroxyphenyl)acetonitrile below 4% area, cooled to 20-25° C. and the phases were allowed to settle. The lower aqueous layer was separated and the organic layer was washed with aqueous sodium hydroxide (10% w/v, 1000 ml, 10 rel vol) followed by aqueous sodium chloride (20% w/v, 1000 ml, 10 rel vol). The resulting organic layer was concentrated to 3-4 relative volumes under reduced pressure. Dimethylsulfoxide (1000 ml, 10 rel vol) was added to the concentrated mass and the distillation was continued to remove residual 2-MeTHF. 2-[2-[2-[benzyl(methyl)amino]ethoxy]phenyl]acetonitrile was isolated as a 13.3% w/w pale brown solution in dimethylsulfoxide (1315 g at 100% strength) with 94.3% purity and 84.7% yield.

¹H-NMR (δ, CDCl₃, 400 MHz): 7.36-7.25 (m, 7H), 6.95 (t, 15.04 Hz, 1H), 6.85 (d, 8.0 Hz, 1H), 4.12 (t, 12.04 Hz, 2H), 3.66 (s, 2H), 3.61 (s, 2H), 2.85 (t, 11.52 Hz, 2H), 2.35 (s, 1H).

¹³C-NMR (δ, CDCl₃, 100.6 MHz): 156.03, 138.85, 129.47, 129.1, 129.0, 128.34, 127.18, 120.89, 118.81, 118.09, 111.38, 66.75, 62.87, 55.78, 43.08, 18.7.

### Step 2: 1-[2-[2-[Benzyl(methyl)amino)ethoxy]phenyl]cyclopropane-carbonitrile

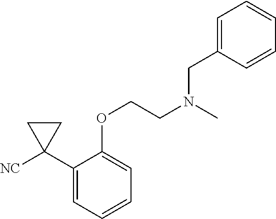 [SMILES: CN(CCOc1ccccc1C1(C#N)CC1)Cc1ccccc1]

A solution of 2-[2-[2-[benzyl(methyl)amino]ethoxy]phenyl]acetonitrile in DMSO (138.9 g, 10.8% w/w, 15.0 g at 100% w/w, 0.49 moles, 1.0 eqv) was further diluted with DMSO (60 mL), followed by addition of sodium hydroxide powder (5.35 g, 2.5 eqv). The resulting suspension was stirred before a solution of ethylene sulphate (8.66 g, 1.3 eqv) in sulfolane (37.5 mL, 2.5 rel vol) was added dropwise over a period of 2 h at 20-25° C. The mixture was stirred and further sodium hydroxide powder was added (2.21 g, 1.0 mol eqv). The mixture was stirred at ambient temperature for 16 h, followed by addition of MTBE (135 mL, 9.0 rel vol) and water (150 mL, 10.0 rel vol). The two-phase mixture was then allowed to settle and the lower aqueous phase was separated and re-extracted with more MTBE. The combined MTBE extracts were washed twice with 10% w/v aqueous sodium chloride solution. 1-[2-[2-[benzyl(methyl)amino) ethoxy]phenyl]cyclopropanecarbonitrile was isolated as a 6.9% w/w solution in methyl tert-butyl ether (12.3 g at 100% strength) with 75% yield.

¹H-NMR (δ, CDCl₃, 400 MHz): 7.36-7.2 (m, 7H), 6.91-6.84 (m, 2H), 4.19 (t, 11.56 Hz, 2H), 3.66 (s, 2H), 2.98 (t, 12.04 Hz, 2H), 2.92 (s, 3H), 1.58-1.55 (m, 2H), 1.26-1.23 (m, 2H).

¹³C-NMR (δ, CDCl₃, 100.6 MHz): 158.23, 139.12, 129.91, 128.97, 128.31, 127.06, 124.1, 123.1, 120.44, 111.63, 67.21, 62.91, 56.11, 43.02, 15.29, 10.31.

### Step 3: 1-[2-[2-[Benzyl(methyl)amino)ethoxy]phenyl]cyclopropane-carboxamide

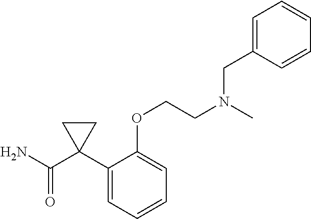 [SMILES: CN(CCOc1ccccc1C1(C(N)=O)CC1)Cc1ccccc1]

A solution of 1-[2-[2-[benzyl(methyl)amino)ethoxy]phenyl]cyclopropanecarbonitrile in methyl tert-butyl ether (1563 g, 6.4% w/w, 93.4 g at 100% w/w, 0.33 moles, 1.0 eq) was concentrated to 3-4 relative volumes under reduced pressure. Tert-butyl alcohol (1500 ml, 15 rel vol) was added and the distillation was continued till the combined reaction mass reached approx. 10 relative volumes. The resulting mixture was cooled to 40° C.; potassium hydroxide powder (64.65 g, 0.98 moles, 3.0 eq) was added in one lot and the reaction mixture was heated to 78-80° C. The reaction mixture was refluxed for 3 h and monitored by HPLC for completion (unreacted 1-[2-[2-[benzyl(methyl)amino)ethoxy]phenyl]cyclopropane-carbonitrile below 3% area). The mixture was cooled to 40° C., quenched with water (800 ml, 8 rel vol) and stirred for 10 minutes. Agitation was stopped and the phases were allowed to settle. The lower aqueous phase was run off and the organic layer concentrated to 3-4 relative volumes under reduced pressure. The concentrated mixture was cooled to 40° C., diluted with isopropyl acetate (1000 ml, 10 rel vol) and washed with aqueous sodium bicarbonate (5% w/v, 800 ml, 8 rel vol) followed by aqueous sodium chloride (5% w/v, 800 ml, 8 rel vol) at 40° C. The organic layer was concentrated to 3-4 relative volumes under reduced pressure, cooled to 40° C. and seeded with 1-[2-[2-[benzyl(methyl)-amino)-ethoxy]phenyl]cyclopropane carboxamide (0.1 g, 0.1% w/w) (appropriate seeds can be generated from the batch by transferring a proportion of the solution into a secondary vessel and either cool this below the point of supersaturation to induce spontaneous crystallization or by evaporation of the solvent). After stirring at 40° C. for 15 min the mixture was cooled to 20-25° C. over a period of 1 h. Heptane (1200 ml, 12 rel vol) was added drop wise over a period of 2 h whilst stirring was maintained at 20-25° C. for 4 h. The suspension was then filtered and washed with heptane (200 ml, 2 rel vol). The wet product was dried at 45° C. in a vacuum oven for 15 h. 1-[2-[2-[benzyl(methyl)amino)ethoxy]phenyl]cyclopropanecarboxamide was isolated as a pale yellow crystalline solid in 96.7% w/w strength (92 g, 98.9% purity, 90.0% yield).

¹H-NMR (δ, CDCl₃, 400 MHz): 7.33-7.24 (m, 7H), 6.93 (t, 14.52 Hz, 1H), 6.87 (d, 8.52 Hz, 1H), 5.55 (s, 1H), 5.18 (s, 1H), 4.15 (t, 11.52 Hz, 2H), 3.61 (s, 2H), 2.88 (t, 11.56 Hz, 2H), 2.33 (s, 3H), 1.61-1.58 (m, 2H), 1.02-0.99 (m, 2H).

¹³C-NMR (δ, CDCl₃, 100.6 MHz): 176.42, 158.17, 139.98, 131.96, 129.3, 128.96, 128.30, 127.06, 120.77, 111.65, 66.90, 62.86, 56.05, 42.94, 26.06, 16.08.

### Step 4: 1-[2-[2-[Benzyl(methyl)amino]ethoxy]phenyl]cyclopropan-amine di para-toluenesulfonic acid

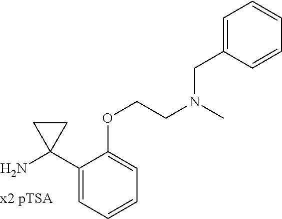 [SMILES: CN(CCOc1ccccc1C1(N)CC1)Cc1ccccc1]

1-[2-[2-[benzyl(methyl)amino]ethoxy]phenyl]cyclopropanecarboxamide (50 g, 0.15 moles, 1.0 eq) was suspended in 2-methyltetrahydrofuran (250 ml, 5 rel vol) and diisopropylethylamine (24.39 g, 0.19 moles, 1.2 eq) was added in one lot, followed by a solution of [bis(trifluoroacetoxy)iodo]benzene (93.73 g, 0.22 moles, 1.4 eq) dissolved in 2-methyltetrahydrofuran (250 ml, 5 rel vol) drop wise over a period of 2 h. The resulting mixture was stirred at 20-25° C. for 2 h and monitored for reaction progress by HPLC for completion (unreacted 1-[2-[2-[benzyl(methyl)amino)-ethoxy]phenyl]-cyclopropane-carboxamide below 2% area). The reaction mixture was warmed to 28° C. and quenched with a solution of concentrated hydrochloric acid (128.44 g, 1.23 moles, 8 eq) dissolved in demineralised water (200 ml, 4 rel vol) drop wise over a period of 1 h. The resulting mixture was stirred at 28° C. for another 1 h and the layers were allowed to settle and separate. The aqueous layer was collected and washed with 2-methyltetrahydrofuran (150 ml, 3 rel vol). 2-methyltetrahydrofuran (250 ml, 5 rel vol) was added to the resulting aqueous layer and the pH was adjusted to 9.5-10.5 using aqueous sodium hydroxide (20% w/v, 150.7 ml, 5.8 eq). The two layers were separated and the resulting aqueous layer was back extracted with 2-methyltetrahydrofuran (250 ml, 5 rel vol). The combined organic layers were washed with aqueous sodium chloride (20% w/v, 250 ml, 5 rel vol) and concentrated to approx. 5 relative volumes under reduced pressure. The reaction mixture was cooled to 20-25° C. and seeded with 1-[2-[2-[benzyl(methyl)amino]ethoxy]phenyl]cyclopropanamine di-pTSA (0.30 g, 0.5% w/w) (the initial seed particles were made following the same process except that pTSA solution (in isopropyl alcohol) was directly added to the concentrated 2-Me THF solution in the absence of any seed and then agitated to precipitate out the di-pTSA salt). The mass was stirred for 10 min at 20-25° C. and a solution of p-toluenesulfonic acid monohydrate (101.7 g, 0.52 moles, 3.4 eq) in isopropyl alcohol (125 ml, 2.5 rel vol) was added drop wise at 20-25° C. over a period of 2 h. The resulting slurry was stirred at 20-25° C. for approx. 7 h and filtered. The filter cake was washed with 2-methyltetrahydrofuran (150 ml, 3 rel vol) and dried at 45° C. in a vacuum oven for approx 3 h. 1-[2-[2-[benzyl (methyl)amino)ethoxy]phenyl]cyclopropanamine di-pTSA was isolated as an off white crystalline solid in 95% w/w strength (78 g, 99.0% purity, 75.0% yield).

¹H-NMR (δ, DMSO, 400 MHz): 9.75 (s, 1H), 8.26 (s, 1H), 7.60-7.36 (m, 11H), 7.13-7.11 (m, 5H), 7.02 (t, 15.04 Hz, 1H), 4.69-4.32 (m, 4H), 3.69 (s, 2H), 2.85 (s, 3H), 2.29 (s, 6H), 1.26-1.21 (m, 2H), 1.14-0.97 (m, 2H).

¹³C-NMR (δ, DMSO, 100.6 MHz): 156.9, 144.8, 138.2, 131.1, 130.8, 130.7, 130.1, 129.6, 128.9, 128.2, 125.4, 124.5, 120.9, 111.9, 62.6, 59.1, 54.1, 33.3, 20.7, 10.9.

### Example 2: Preparation of 1-[2-[2-[benzyl(methyl)amino)ethoxy]phenyl]cyclopropan-amine (Scheme 6)

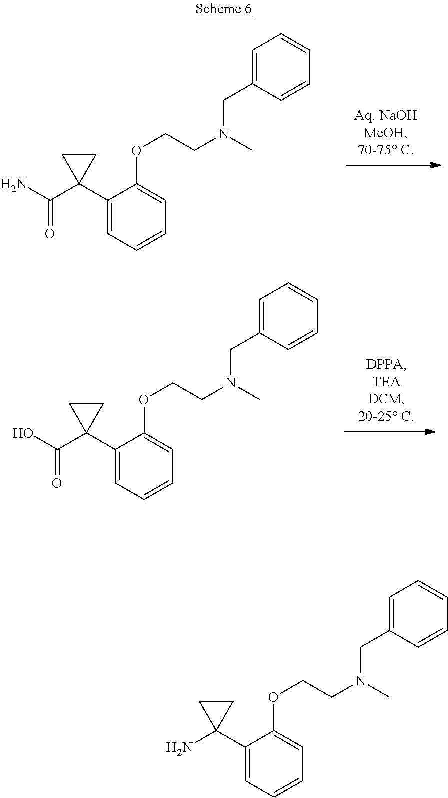 [SMILES: CN(CCOc1ccccc1C1(C(N)=O)CC1)Cc1ccccc1 ; CN(CCOc1ccccc1C1(C(=O)O)CC1)Cc1ccccc1 ; CN(CCOc1ccccc1C1(N)CC1)Cc1ccccc1]

### Step 1: 1-[2-[2-Benzyl(methyl)amino]ethoxy]phenyl]cyclopropane carboxylic acid

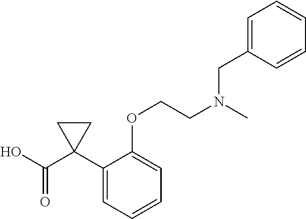 [SMILES: CN(CCOc1ccccc1C1(C(=O)O)CC1)Cc1ccccc1]

To a solution of 1-[2-[2-benzyl(methyl)amino]ethoxy]phenyl]cyclopropane carboxamide (Example 1, step 3) (5 g, 0.013 moles, 1.0 eq) in methanol (25 ml, 5 rel vol), aqueous sodium hydroxide (10% w/v, 22.35 ml, 5.0 eq) was added followed by tetrabutylammonium hydroxide (4.35 ml, 0.007 moles, 0.5 eq), and the reaction mixture was heated to reflux. After 19 h, another lot of aqueous sodium hydroxide (10% w/v, 22.35 ml, 5.0 eq) was added, and stirring at 65-70° C. was continued for further 42 h. Additional aqueous sodium hydroxide (10% w/v, 22.35 ml, 5.0 eq) and methanol (25 ml, 5 rel vol) were added, and the mixture was stirred for further 66 h. The reaction was monitored by HPLC for completion (2-(1-[2-[2-benzyl(methyl)amino]ethoxy]phenyl]cyclopropanecarboxamide below 4% area). The reaction mixture was concentrated to approx. 10 relative volumes, then diluted with 2-methyltetrahydrofuran (50 ml, 10) before the layers were allowed to settle and separate. After phase separation the organic layer was washed with water (50 ml, 10 rel vol). The pH of the combined aqueous layers was adjusted to approx. 5 and the desired product was extracted into 2-methyltetrahydrofuran (50 ml, 10 rel vol). The organic layer was concentrated to dryness under reduced pressure. 1-[2-[2-benzyl(methyl)-amino]ethoxyl-phenyl]cyclopropanecarboxylic acid was isolated as a pale yellow solid (2.4 g, 96.1% purity, 55.6% yield).

¹H-NMR (δ, CDCl₃, 400 MHz): 7.29-7.16 (m, 7H), 6.88 (t, 15.04 Hz, 1H), 6.75 (d, 8.04 Hz, 1H), 4.14 (t, 10.52 Hz, 2H), 3.76 (s, 2H), 2.95 (t, 10.04 Hz, 2H), 2.40 (s, 3H) 1.53-1.52 (m, 2H), 1.02-1.01 (m, 2H).

¹³C-NMR (δ, CDCl₃, 100.6 MHz): 178.8, 157.9, 134.9, 130.5, 130.1, 129.9 128.5, 128.0, 120.6, 111.4, 65.1, 61.3, 54.8, 41.4, 25.6, 16.1.

### Step 2: 1-[2-[2-Benzyl(methyl)amino]ethoxy]phenyl]cyclopropanamine

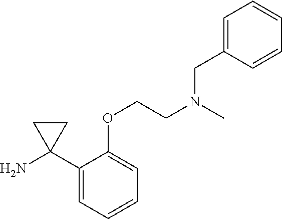 [SMILES: CN(CCOc1ccccc1C1(N)CC1)Cc1ccccc1]

To a solution of 1-[2-[2-benzyl(methyl)amino]ethoxy]phenyl]cyclopropanecarboxylic acid (1.0 g, 0.003 moles, 1.0 eq) in dichloromethane (15 ml, 15 rel vol) at 20-25° C., diphenylphosphoryl azide (DPPA, 0.66 ml, 0.003 moles, 1.0 eq) was added followed by triethylamine (0.51 ml, 0.004 moles, 1.2 eq). The reaction mixture was stirred for 2 h at 20-25° C., and then monitored by HPLC for completion (1-[2-[2-benzyl(methyl)amino]ethoxy] phenyl]cyclopropanecarboxylic acid below 4% area). The reaction was quenched with aqueous ammonium chloride (20% w/v, 10 ml, 10 rel vol) and the layers were allowed to separate. After phase split the organic layer was diluted with toluene (10 ml, 10 rel vol). The organic phase was then concentrated to approx. 10 relative volumes under reduced pressure before charging dilute hydrochloric acid (10 ml, 0.012 moles, 4.0 eq). The reaction mixture was heated to 65-70° C. and stirred for 18 h. The reaction mixture was then cooled to 20-25° C. and the phases were separated. The aqueous layer was basified with aqueous sodium hydroxide (10% w/v, 4 ml, 4 rel vol) and the desired product was extracted into methyl-tert-butyl ether (20 ml, 20 rel vol) and concentrated to complete dryness under reduced pressure. 1-[2-[2-benzyl(methyl)amino]ethoxy]phenyl]cyclopropanamine was isolated as a pale yellow oil (0.34 g, 98.5% purity, 37.3% yield).

¹H-NMR (δ, CDCl₃, 400 MHz): 7.33-7.16 (m, 7H), 6.88-6.82 (m, 2H), 4.16 (t, 11.04 Hz, 2H), 3.63 (s, 2H), 2.89 (t, 11.52 Hz, 2H), 2.34 (s, 3H) 0.94-0.91 (m, 2H), 0.81-0.79 (m, 2H).

¹³C-NMR (δ, CDCl₃, 100.6 MHz): 157.93, 138.87, 134.24, 129.03, 128.35, 128.35, 127.97, 127.14, 120.40, 111.47, 66.38, 62.95, 56.32, 42.88, 35.08, 14.11.

### Example 3: Preparation of benzyl N-[2-[2-(1-aminocyclopropyl)phenoxy]ethyl]-N-methyl-carbamate para-toluenesulfonic acid (Scheme 7)

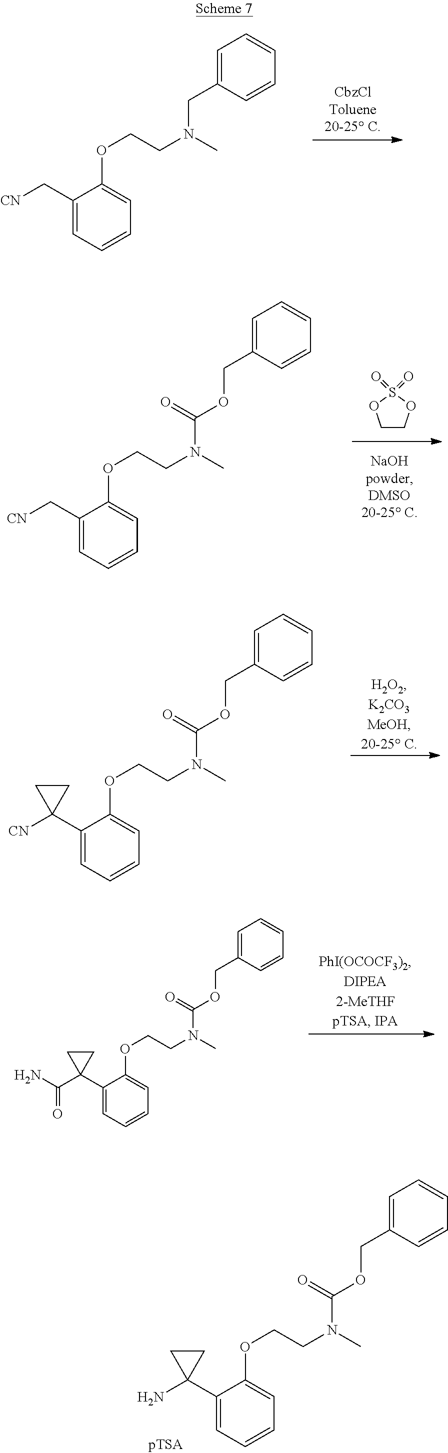

### Step 1: Benzyl N-[2-[2-(cyanomethyl)phenoxy]ethyl]-N-methyl-carbamate

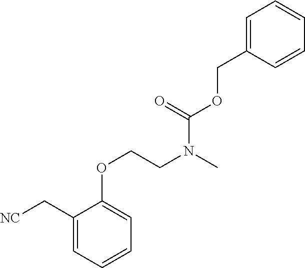 [SMILES: CN(CCOc1ccccc1CC#N)C(=O)OCc1ccccc1]

To a solution of 2-[2-[2-[benzyl(methyl)amino]ethoxy]phenyl]acetonitrile (Example 1, step 1) (5 g, 0.02 moles, 1.0 eq) in toluene (50 ml, 10 rel vol), benzyl chloroformate (2.89 ml, 0.02 moles, 1.2 eq) was added drop wise at 20-25° C., and the resulting mass was stirred for 4 h. Another lot of benzyl chloroformate (1.21 ml, 0.01 moles, 0.5 eq) was then charged and continued to stir the reaction mass for further 3 h. The reaction was monitored by HPLC for completion (2-[2-[2-[benzyl(methyl)amino]ethoxy]phenyl]acetonitrile below 4% area), then quenched with aqueous sodium bicarbonate (5% w/v, 50 ml, 10 rel vol) and allowed the layers to settle. Separated the two layers and washed the resulting organic layer with water (50 ml, 10 rel vol). The organic layer was concentrated to complete dryness under reduced pressure. Washed the residue (yellow oil) twice with hexane (25 ml, 5 rel vol) and stirred the resulting oil in hexane (25 ml, 5 rel vol) for 2 h at 20-25° C. to obtain benzyl N-[2-[2-(cyanomethyl)phenoxy]ethyl]-N-methyl-carbamate as a pale yellow solid (3.9 g, 93.1% purity, 77.5% yield).

¹H-NMR (δ, CDCl₃, 400 MHz): 7.36-7.25 (m, 7H), 6.96 (t, 15.04 Hz, 1H), 6.88-6.77 (m, 1H), 5.14 (s, 2H), 4.18-4.09 (m, 2H), 3.73-3.71 (m, 2H), 3.62-3.56 (m, 2H) 3.07 (s, 3H).

¹³C-NMR (δ, CDCl₃, 100.6 MHz) mixture of rotamers: 156.44, 155.87, 155.69, 136.69, 133.55, 129.61, 129.42, 128.51, 128.12, 127.90, 121.22, 121.14, 118.65, 117.92, 111.12, 67.42, 67.24, 66.35, 66.24, 48.91, 48.14, 36.08, 35.83, 18.77.

### Step 2: Benzyl N-[2-[2-(1-cyanocyclopropyl)phenoxy]ethyl]-N-methyl-carbamate

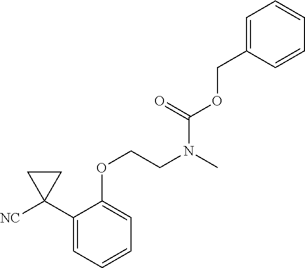 [SMILES: CN(CCOc1ccccc1C1(C#N)CC1)C(=O)OCc1ccccc1]

To a solution of benzyl N-[2-[2-(cyanomethyl)phenoxy]ethyl]-N-methyl-carbamate (2 g, 0.006 moles, 1.0 eq) in dimethylsulfoxide (20 ml, 10 rel vol), powder sodium hydroxide (0.59 g, 0.014 moles, 2.5 eq) was added and the resulting mass was stirred at 20-25° C. for 10 min. A solution of 1,3,2-dioxathiolane-2,2-dioxide (1.09 g, 0.009 moles, 1.5 eq) in tetrahydrofuran (4 ml, 4 rel vol) was then added drop wise to the reaction mass at 20-25° C. and stirred the resulting mass for 1 h. Another lot of powder sodium hydroxide (0.24 g, 0.006 moles, 1.0 eq) was charged and continued to stir the reaction mass for further 15 h. The reaction was monitored by HPLC for completion (N-[2-[2-(cyanomethyl)phenoxy]ethyl]-N-methyl-carbamate below 4% area). The reaction mass was quenched with methyl-tert-butyl ether (20 ml, 10 rel vol) followed by water (20 ml, 10 rel vol), and allowed the layers to settle. Separated the two layers and washed the resulting organic layer with aqueous sodium chloride (5% w/v, 20 ml, 10 rel vol). The organic layer was then concentrated down to complete dryness under reduced pressure to obtain benzyl N-[2-[2-(1-cyanocyclopropyl)phenoxy]ethyl]-N-methyl-carbamate as pale yellow oil (1.7 g, 91.2% purity, 84.6% yield).

¹H-NMR (δ, CDCl₃, 400 MHz) mixture of rotamers: 7.37-7.15 (m, 7H), 6.90-6.76 (m, 2H), 5.15, 5.13 (s, 2H, rotamers), 4.21-4.13 (m, 2H), 3.78-3.74 (m, 2H), 3.16, 3.14 (s, 3H, rotamers), 1.53 (m, 2H), 1.22-1.17 (m, 2H).

¹³C-NMR (δ, CDCl₃, 100.6 MHz) mixture of rotamers: 157.98, 156.36, 156.03, 136.75, 129.97, 129.82, 128.42, 128.12, 127.80, 126.79, 123.34, 122.88, 120.69, 111.52, 67.23, 67.07, 66.87, 49.09, 48.25, 36.32, 36.12, 15.14, 10.18.

### Step 3: Benzyl N-[2-[2-(1-carbamoylcyclopropyl)phenoxy]ethyl]-N-methyl-carbamate

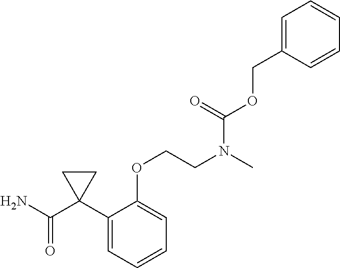 [SMILES: CN(CCOc1ccccc1C1(C(N)=O)CC1)C(=O)OCc1ccccc1]

A solution of benzyl N-[2-[2-(1-cyanocyclopropyl)phenoxy]ethyl]-N-methyl-carbamate (1 g, 0.003 moles, 1.0 eq) in methanol (10 ml, 10 rel vol) was cooled to 0-5° C. and potassium carbonate (0.43 g, 0.003 moles, 1.2 eq) was added to it. Aqueous hydrogen peroxide (30% w/w, 1.32 ml, 0.013 moles, 5.0 eq) was then charged and the resulting reaction mass was gradually warmed to 20-25° C. The reaction mass was stirred for 17 h at 20-25° C. and the reaction was monitored by HPLC for completion (benzyl N-[2-[2-(1-cyanocyclopropyl) phenoxy]ethyl]-N-methyl-carbamate below 4% area). The resulting mass was quenched with water (8 ml, 8 rel vol) followed by ethyl acetate (10 ml, 10 rel vol) and allowed the layers to settle. Separated the two layers and washed the organic layer with aqueous sodium chloride (5% w/v, 10 ml, 10 rel vol). The resulting organic layer was concentrated to complete dryness under reduced pressure to give the residue (pale yellow oil), which was then stirred in methyl-tert-butyl ether (10 ml, 10 rel vol) for 2 h at 20-25° C. to obtain benzyl N-[2-[2-(1-carbamoylcyclopropyl)phenoxy]ethyl]-N-methyl-carbamate as an off white solid (260 mg, 99.4% purity, 28.0% yield).

¹H-NMR (δ, CDCl₃, 400 MHz): 7.35-7.24 (m, 7H), 6.97-6.81 (m, 2H), 5.38-5.35 (m, 2H), 5.13 (s, 2H), 4.18-4.08 (m, 2H), 3.96-3.68 (m, 2H), 3.09 (s, 3H), 1.60-1.59 (m, 2H), 1.01-1.00 (m, 2H).

¹³C-NMR (δ, CDCl₃, 100.6 MHz) mixture of rotamers: 176.41, 176.33, 158.10, 156.40, 156.00, 136.78, 131.82, 129.46, 128.50, 128.06, 127.84, 121.14, 121.05, 111.63, 67.27, 67.08, 67.03, 66.95, 49.31, 48.42, 36.56, 36.39, 25.99, 16.10.

### Step 4: Benzyl N-[2-[2-(1-aminocyclopropyl)phenoxy]ethyl]-N-methyl-carbamate para-toluenesulfonic acid

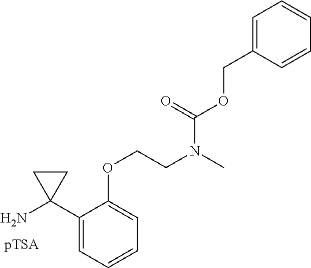 [SMILES: CN(CCOc1ccccc1C1(N)CC1)C(=O)OCc1ccccc1]

To a mixture of benzyl N-[2-[2-(1-carbamoylcyclopropyl)phenoxy]ethyl]-N-methyl-carbamate (1 g, 0.002 moles, 1.0 eq) and diisopropylethylamine (0.53 ml, 0.003 moles, 1.2 eq) in 2-methyltetrahydrofuran (5 ml, 5 rel vol), a solution of [bis(trifluoroacetoxy)iodo]benzene (1.66 g, 0.004 moles, 1.5 eq) in 2-methyltetrahydrofuran (5 ml, 5 rel vol) was added drop wise at 20-25° C. The resulting reaction mass was stirred for 1 h at 20-25° C. and the reaction was monitored by HPLC for completion (benzyl N-[2-[2-(1-carbamoylcyclopropyl)phenoxy]ethyl]-N-methyl-carbamate below 4% area). The reaction mass was then quenched with a solution of hydrochloric acid (36.5% w/w, 1.72 ml, 0.02 moles, 8.0 eq) in water (5 ml, 5 rel vol) and allowed the layers to settle. Separated the two layers and washed the resulting organic layer with aqueous sodium hydroxide (10% w/v, 5 ml, 5 rel vol) and concentrated down to complete dryness under reduced pressure. The residue (pale brown oil) was washed twice with hexane (20 ml, 20 rel vol) and once again distilled to complete dryness under reduced pressure. Redissolved the residue in 2-methyltetrahydrofuran (5 ml, 5 rel vol) and charged a solution of p-toluenesulfonic acid monohydrate (0.48 g, 0.002 moles, 1.0 eq) in 2-methyltetrahydrofuran (2.5 ml, 2.5 rel vol) drop wise into it. The resulting mass was stirred for 1 h at 20-25° C. and then concentrated down to 2 relative volumes under reduced pressure. Stirred the resulting mass in isopropyl alcohol (10 ml, 10 rel vol) for 1 h at 20-25° C. and then filtered to obtain benzyl N-[2-[2-(1-aminocyclopropyl)phenoxy]ethyl]-N-methyl-carbamate pTSA salt as a white solid (90 mg, 97.6% purity, 10.6% yield).

¹H-NMR (δ, CDCl₃, 400 MHz) mixture of rotamers: 8.23-8.21 (m, 3H), 7.49 (d, 8.04 Hz, 2H), 7.36-7.33 (m, 7H), 7.13-6.94 (m, 4H), 5.11, 5.08 (s, 2H, rotamers), 4.19 (t, 10.52 Hz, 2H), 3.74 (s, 2H), 3.01, 2.99 (s, 3H, rotamers), 2.28 (s, 3H) 1.24 (m, 2H), 1.04-1.01 (m, 2H).

¹³C-NMR (δ, CDCl₃, 100.6 MHz) mixture of rotamers: 157.59, 156.00, 155.65, 145.38, 137.80, 136.84, 130.62, 128.40, 128.09, 127.80, 127.12, 125.46, 124.35, 120.37, 111.56, 66.29, 65.75, 47.96, 47.42, 35.64, 34.70, 33.64, 21.26, 20.74, 10.73.

### Example 4: Preparation of 3-(3-bromo-2-oxo-pyrazin-1-yl)-N-cyclopropyl-5-fluoro-4-methyl-benzamide (Scheme 8)

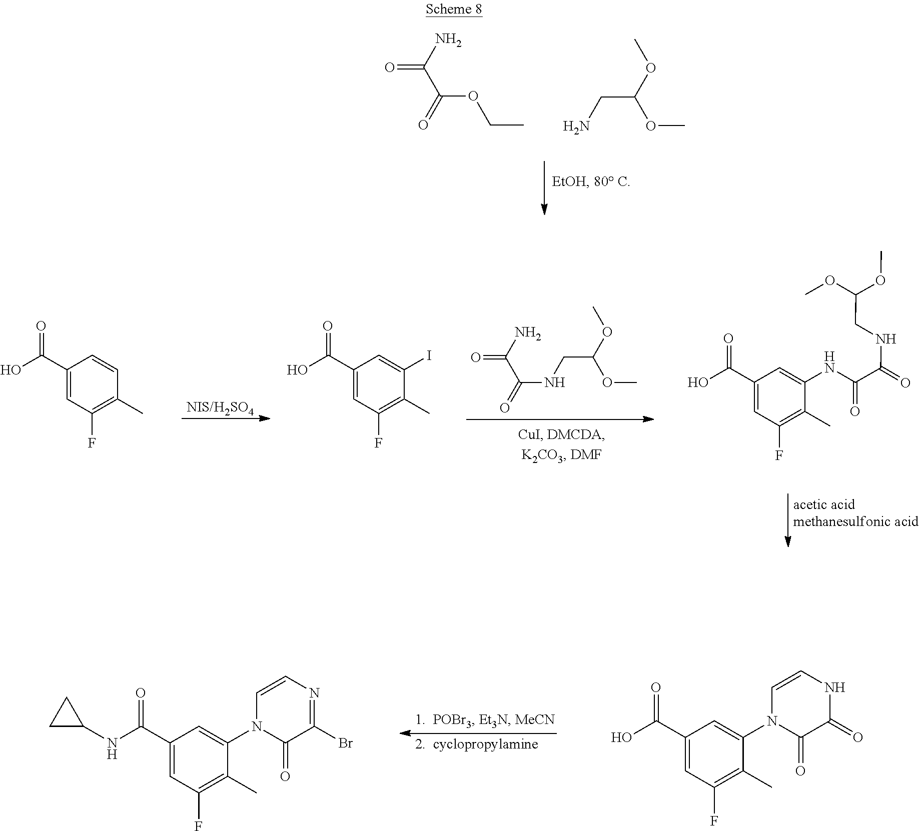

### Step 1: N′-(2,2-dimethoxyethyl)oxamide

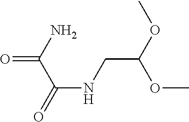

A solution of 2,2-dimethoxyethanamine (36.8 mL, 334 mmol) in ethanol (40 mL, 1 rel vol) was charged over a period of 10 min to a warmed (50° C.) suspension of ethyl 2-amino-2-oxo-acetate (40 g, 334.76 mmol) in ethanol (320 mL, 8 rel vol). The reaction was heated to reflux (80° C.) and the reaction progress was monitored by HPLC. Solids precipitated in the course of the reaction. Once complete, the reaction mixture was cooled gradually to ambient temperature over 4 h. The solid product was filtered, washed with ethanol and dried in a vacuum oven at 60° C. over night to leave N′-(2,2-dimethoxyethyl)oxamide as a white fluffy solid (53.5 g, 99.1% purity, 90% yield).

¹H-NMR (δ, DMSO, 500 MHz): 3.24 (8H, m), 4.49 (1H, t), 7.80 (1H, s), 8.10 (1H, s), 8.60 (1H, s).

¹³C-NMR (δ, CDCl₃, 101 MHz): 40.9, 53.4, 101.6, 160.8, 162.4.

### Step 2: 3-fluoro-5-iodo-4-methyl-benzoic acid

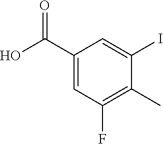

To 3-fluoro-4-methyl benzoic acid (60 g, 389 mmol, 1.0 eqv) was added concentrated sulfuric acid (210 ml, 3.5 rel vol) The mixture was stirred at ambient temperature for approximately 15 minutes and was then cooled to between −5° C. and −10° C. A solution of N-iodosuccinimide (NIS, 140.1 g, 623 mmol, 1.6 eqv) in conc. sulfuric acid (360 ml) was added slowly, maintaining the internal temperature in the range −5° C. to −10° C. The mixture was stirred for a further 2 h at this temperature and monitored for complete conversion. The reaction mixture was added to water (1.8 L, 30 rel vol) maintaining the temperature below 30° C. The resulting suspension was filtered and the filter cake was slurried in 5% aq. Na₂SO₃ solution (600 mL, 10 rel vol). After filtration and recrystallization from toluene (90 mL, 1.5 rel vol), 3-fluoro-5-iodo-4-methyl-benzoic acid (71.9 g, 66% yield) was isolated as a white solid.

¹H-NMR (δ, DMSO, 400 MHz): 2.3 (d, 3H), 7.6 (dd, 1H), 8.0-8.3 (m, 1H).

¹³C-NMR (δ, DMSO, 101 MHz): 20.4, 102.7, 116.0, 132.1, 133.7, 135.5, 159.3, 165.3.

### Step 3: 3-[[2-(2,2-dimethoxyethylamino)-2-oxo-acetyl]amino]-5-fluoro-4-methyl-benzoic acid

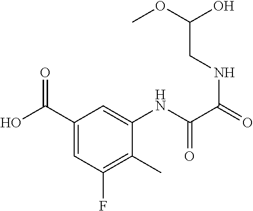 [SMILES: COC(O)CNC(=O)C(=O)Nc1cc(C(=O)O)cc(F)c1C]

3-fluoro-5-iodo-4-methyl-benzoic acid (40 g, 141.41 mmol, 1.0 eqv), N′-(2,2-dimethoxyethyl)oxamide (30.2 g, 170 mmol, 1.2 eqv) and potassium carbonate (39.1 g, 283 mmol, 2.0 eqv) were placed into a 500 mL jacketed vessel fitted with condenser, nitrogen/vacuum inlet and overhead stirrer. DMF (320 mL, 8 rel vol) was added and the mixture was stirred vigorously to keep all solids suspended. Trans-(1R,2R)—N,N′-bismethyl-1,2-cyclohexanediamine (7.04 mL, 42.4 mmol, 0.3 eqv) was charged and the mixture was degassed by sub-surface sparging via a needle. The mixture was heated to 50° C. before iodocopper (8.24 g, 42.4 mmol, 0.3 eqv) was charged to the suspension. The mixture was stirred at 110° C. under an atmosphere of nitrogen for 19 h and was then cooled to ambient temperature. To the dark green suspension was charged at 20° C. ethanol (80 mL, 2 rel vol) and the contents of the vessel was discharged slowly to a 2 L beaker filled with 1M citric acid (640 mL, 16 rel vol). The resulting slurry was diluted with water (200 mL, 5 rel vol) and filtered. The filter cake was washed with water (4×200 mL) and dried in a vacuum oven at 60° C. over night to give 3-[[2-(2,2-dimethoxyethylamino)-2-oxo-acetyl]amino]-5-fluoro-4-methyl-benzoic acid (36.42 g, 98.9% purity, 77% yield) as a white solid.

¹H-NMR (δ, DMSO, 500 MHz): 2.16 (3H, s), 3.28 (6H, s), 3.33 (2H, t), 4.55 (1H, t), 7.53 (1H, d), 7.89 (1H, s), 8.92 (1H, t), 10.46 (1H, s), 13.24 (1H, s).

### Step 4: 3-(2,3-dioxo-1H-pyrazin-4-yl)-5-fluoro-4-methyl-benzoic acid

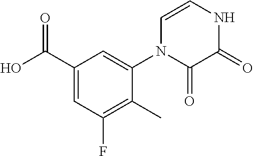 [SMILES: Cc1c(F)cc(C(=O)O)cc1-n1cc[nH]c(=O)c1=O]

3-[[2-(2,2-dimethoxyethyl amino)-2-oxo-acetyl]amino]-5-fluoro-4-methyl-benzoic acid (35.00 g, 102 mmol, 1.0 eqv) was suspended at ambient temperature in acetic acid (350 mL, 10 rel vol) to give a thick suspension. Methanesulfonic acid (6.67 ml, 102 mmol, 1.0 eqv) was added in one portion and the suspension was heated at 110° C. for 15 h. After cooling the mixture to 20° C., ethyl acetate (101 ml, 2.9 rel vol) was charged and the reaction stirred at 20° C. for 4 h. The precipitating solids were collected by filtration and were washed with ethyl acetate (100 ml, 3 rel vol), water (170 ml, 5 rel vol) and dried in a vacuum oven at 50° C. over the weekend to yield 3-(2,3-dioxo-1H-pyrazin-4-yl)-5-fluoro-4-methyl-benzoic acid (25.0 g, 96.0% purity, 88.8% yield) as a grey solid.

¹H-NMR (δ, DMSO, 500 MHz): 2.07 (3H, d), 6.40-6.52 (2H, m), 7.70-7.77 (2H, m), 11.31-11.47 (1H, m), 13.34 (1H, s).

¹³C-NMR (δ, DMSO, 101 MHz): 10.62, 110.12, 113.99, 116.17, 124.91, 128.33, 131.20, 140.84, 156.24, 156.44, 160.74, 165.94.

### Step 5: 3-(3-bromo-2-oxo-pyrazin-1-yl)-N-cyclopropyl-5-fluoro-4-methyl-benzamide

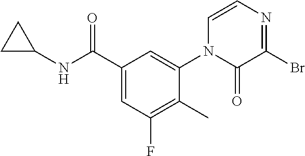 [SMILES: Cc1c(F)cc(C(=O)NC2CC2)cc1-n1ccnc(Br)c1=O]

Triethylamine (5.10 mL, 36.6 mmol, 1.0 eqv) was added to a suspension of 3-(2,3-dioxo-1H-pyrazin-4-yl)-5-fluoro-4-methyl-benzoic acid (10.0 g, 36.3 mmol, 1.0 eqv) in acetonitrile (80 mL, 8 rel vol). The mixture was heated to 80° C. To the hot, stirred mixture a solution of phosphorus oxybromide (20.8 g, 72.55 mmol, 2.0 eqv) in acetonitrile (50 mL, 5 rel vol) was added dropwise over 20 minutes and the reaction was stirred at 80° C. for a further 1 h. The mixture was allowed to cool to RT over 30 minutes and then cooled further to −9° C. A solution of cyclopropylamine (15.1 mL, 218 mmol, 6.0 eqv) in acetonitrile (20 mL, 2 rel vol) was added dropwise over 35 minutes maintaining the internal temperature below 0° C., and then stirred at this temperature for a further 30 minutes. The reaction was allowed to warm to ambient temperature and was then quenched with saturated aqueous sodium carbonate solution (200 mL) and diluted with ethyl acetate (500 mL) and water (50 mL). The lower aqueous phase was run off and the upper organic phase was washed with aqueous sodium carbonate (200 mL), aqueous 1M citric acid solution (2×100 mL), water (3×100 mL), and finally with brine (100 mL). After drying over anhydrous magnesium sulfate and filtration, the filtrate was concentrated to give a yellow/brown solid that was subsequently crystallised from hot ethyl acetate. The solid was re-slurried in MTBE (100 mL) overnight, isolated by filtration and air dried for 2 hours to yield 3-(3-bromo-2-oxo-pyrazin-1-yl)-N-cyclopropyl-5-fluoro-4-methyl-benzamide (12.31 g, 88.6% purity, 82.0% yield) as a cream solid.

¹H-NMR (δ, DMSO, 500 MHz): 0.56 (2H, s), 0.64-0.78 (2H, m), 2.03 (3H, s), 2.8-2.9 (1H, m), 7.31 (1H, d), 7.72 (2H, s), 7.79 (1H, d), 8.55 (1H, s).

¹³C-NMR (δ, DMSO, 101 MHz): 5.6, 5.7, 9.9, 23.1, 114.7, 122.1, 122.4, 125.6, 130.1, 134.2, 139.9, 141.6, 151.5, 160.2, 164.8.

### Example 5: Preparation of N-cyclopropyl-3-fluoro-4-methyl-5-[3-[[1-[2-[2-(methylamino)ethoxy]phenyl]cyclopropyl]amino]-2-oxo-1(2H)-pyrazinyl]-benzamide (Scheme 9)

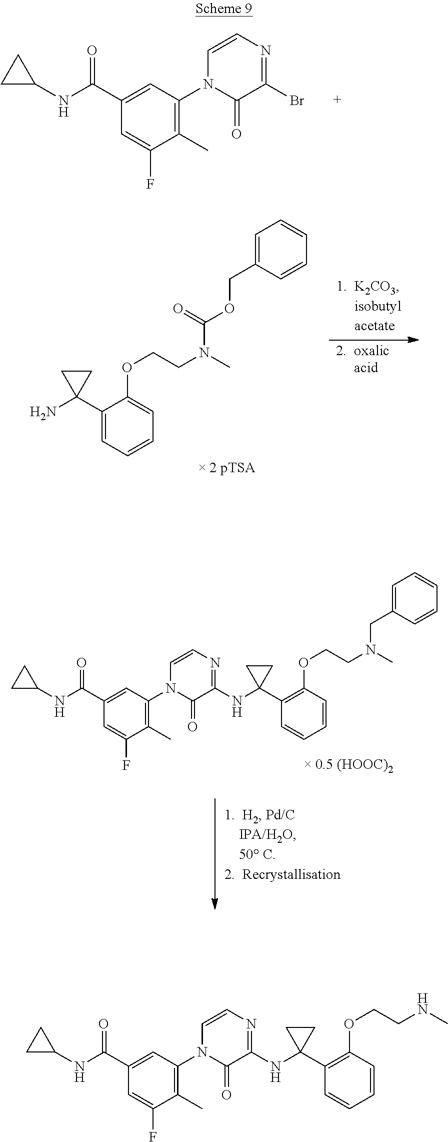 [SMILES: Cc1c(F)cc(C(=O)NC2CC2)cc1-n1ccnc(Br)c1=O ; CN(CCOc1ccccc1C1(N)CC1)C(=O)OCc1ccccc1 ; Cc1c(F)cc(C(=O)NC2CC2)cc1-n1ccnc(NC2(c3ccccc3OCCN(C)Cc3ccccc3)CC2)c1=O ; CNCCOc1ccccc1C1(Nc2nccn(-c3cc(C(=O)NC4CC4)cc(F)c3C)c2=O)CC1]

### Step 1: Benzyl-[2-[2-[1-[[4-[5-(cyclopropylcarbamoyl)-3-fluoro-2-methyl-phenyl]-3-oxo-pyrazin-2-yl]amino]cyclopropyl]phenoxy]ethyl]-methyl-ammonium hemi-oxalate

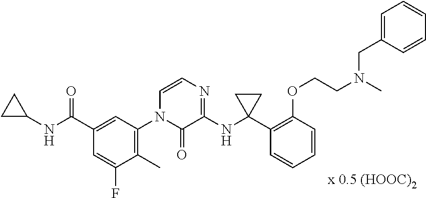 [SMILES: Cc1c(F)cc(C(=O)NC2CC2)cc1-n1ccnc(NC2(c3ccccc3OCCN(C)Cc3ccccc3)CC2)c1=O]

A 250 mL jacketed vessel fitted with condenser, nitrogen inlet, overhead stirrer, internal temperature probe, and oxygen sensor was thoroughly inerted and flushed with nitrogen to reduce oxygen levels down to 0.12%. 3-(3-bromo-2-oxo-pyrazin-1-yl)-N-cyclopropyl-5-fluoro-4-methyl-benzamide (12.00 g, 30.0 mmol, 1.0 eqv) was charged to the vessel as a solid followed by degassed isobutyl acetate (121 mL, 10 rel vol), 1-[2-[2-[Benzyl(methyl)amino]ethoxy]phenyl]cyclopropan-amine di para-toluenesulfonic acid (21.2 g, 33.1 mmol, 1.1 eqv) and potassium carbonate (13.7 g, 99.1 mmol, 3.3 eqv). The mixture was stirred under nitrogen for 30 minutes at ambient temperature until the oxygen level dropped to <0.2% before the internal temperature was set to 110° C. The batch was stirred at 110° C. for 24 hours and then allowed to cool to 40° C. A solution of 0.5M sodium hydroxide (60 mL) was added to the mixture followed by ethanol (8.8 mL, 150 mmol, 5.0 eqv). The mixture was stirred for 5 mins and the phases were allowed to separate. The lower aqueous phase was run off and the upper organic phase was washed with 0.5M sodium hydroxide (60 mL). The basic aqueous phase was run off and 2M hydrochloric acid (60.5 mL) and ethanol (8.8 mL, 150 mmol, 5.0 eqv) was added to the organic layer and the mixture was heated and stirred at 40° C. for 5 mins (the product now resides in the acidic aqueous phase). The organic phase was separated and discarded, and the aqueous phase was washed with isobutyl acetate (2×60 mL). After separating the phases, the organic layer was discarded again and the acidic layer was then added dropwise over 35 min to a warmed (40° C.) mixture of tert-butyl methyl ether (99 mL), 4M sodium hydroxide (33 mL) and ethanol (8.8 mL), followed by the addition of additional tert-butyl methyl ether (60 mL). The mixture was stirred at 40° C. for 5 min and then allowed to separate. The organic phase was retained, the aqueous phase discarded. The organic phase was solvent-swapped into pure ethanol by charging ethanol (60.5 mL) followed by atmospheric distillation until only pure ethanol distills over to a final concentration of crude product in 110 mL (10 rel vol) ethanol. The ethanol solution was stirred at 50° C. and water (49.5 mL, 4.5 rel vol) was added dropwise over 30 minutes. The internal temperature of the now cloudy mixture was raised to 55° C. to give a clear solution prior to seeding with 10 mg product (seeds can be obtained by removing an aliquot of the solution and cooling this to 0-10° C.). The mixture was stirred at 55° C. for 15.5 h before water (5.5 mL, 0.5 rel vol) was charged over 5 minutes. The suspension was then ramp-cooled from 55° C. to 20° C. over 5 h and then temperature held at 20° C. for 30 minutes. The product was filtered and slurry-washed with (1:1) ethanol/water (55 mL, 5 rel vol) at 5° C. for 30 minutes. Filtration and drying in the vacuum oven at 50° C. for 2 days yielded 3-[3-[[1-[2-[2-[benzyl(methyl)amino]ethoxy]phenyl]cyclopropyl]amino]-2-oxo-pyrazin-1-yl]-N-cyclopropyl-5-fluoro-4-methyl-benzamide (12.33 g, 97% purity, 65% yield) as a beige solid.

¹H-NMR (δ, DMSO, 500 MHz): 0.5-0.6 (2H, m), 0.6-0.7 (2H, m), 1.0-1.2 (4H, m), 1.9 (3H, d), 2.2 (3H, s), 2.8-2.9 (2H, m), 2.8-2.9 (1H, m), 3.6 (2H, s), 4.1 (2H, br t), 6.7 (1H, d), 6.8-6.9 (1H), 6.8-6.9 (1H, m), 6.9 (1H, d), 7.1-7.2 (1H, m), 7.2 (1H, d), 7.2-7.3 (2H, m), 7.3 (1H, s), 7.3-7.3 (2H, m), 7.5 (1H, dd), 7.6 (1H, s), 7.7-7.8 (1H, m), 8.4 (1H, d).

¹³C-NMR (δ, DMSO, 127 MHz): 5.6, 5.7, 10.0, 14.0, 14.1, 23.1, 32.8, 42.3, 55.7, 62.0, 66.0, 111.7, 114.3, 117.5, 119.4, 121.7, 121.9, 125.6, 126.8, 128.1, 128.3, 128.7, 129.5, 130.2, 134.0, 139.1, 139.9, 150.1, 151.0, 157.7, 160.3, 164.7.

### Recrystallization to Hemi-Oxalate Salt:

3-[3-[[1-[2-[2-[benzyl(methyl)amino]ethoxy]phenyl]cyclopropyl]amino]-2-oxo-pyrazin-1-yl]-N-cyclopropyl-5-fluoro-4-methyl-benzamide (18.23 g, 28.52 mmol, 1.0 eqv) was dissolved in 2-propanol (83 mL, 4.5 rel vol) and heated to 65° C. To the hot solution was added oxalic acid (1.29 g, 14.3 mmol, 0.5 eqv) and the mixture was allowed to start cooling slowly. At approximately 57-58° C. seeds of the desired hemi-oxalate salt (89 mg, 0.5%; seeds of the hemi-oxalate salt can be obtained by retrieving an aliquot of the hot solution and allowing this to cool to ambient temperature; crystallization will occur spontaneously) were added and the mixture was stirred for 30 minutes whilst cooling to 50° C. The mixture was then allowed to cool to 20° C. over 3 h and stirred at 20° C. overnight (15 h). The product was isolated by filtration and dried in the vacuum oven at 50° C. to yield benzyl-[2-[2-[1-[[4-[5-(cyclopropylcarbamoyl)-3-fluoro-2-methyl-phenyl]-3-oxo-pyrazin-2-yl]amino]cyclopropyl]phenoxy]ethyl]-methyl-ammonium hemi-oxalate (16.56 g, 97.9% purity, 90.8% yield) as a cream solid.

¹H-NMR (δ, DMSO, 500 MHz): 0.5 (2H, m), 0.6-0.7 (2H, m), 1.0-1.1 (1H, m), 1.1-1.2 (3H, m), 1.9 (3H, d), 2.5-2.5 (3H, m), 2.8-2.8 (1H, m), 3.1 (2H, br s), 3.9 (2H, br s), 4.2 (2H, br t), 6.7 (1H, d), 6.8-6.9 (1H, m), 6.8-6.9 (1H, m), 6.9 (1H, d), 7.2 (1H, td), 7.3-7.4 (3H, m), 7.4 (2H, d), 7.4 (1H, s), 7.5 (1H, dd), 7.6 (1H, s), 7.7 (1H, dd), 8.5 (1H, d).

¹³C-NMR (δ, DMSO, 127 MHz): 5.6, 5.7, 10.0, 14.0, 14.1, 23.1, 32.7, 41.2, 55.1, 60.7, 64.7, 111.7, 114.3, 117.5, 119.7, 121.7, 122.0, 125.6, 127.8, 128.4 (m, 3C), 128.4, 129.4 129.7, 130.6, 134.0, 139.9, 150.1, 151.0, 157.4, 160.3, 164.7.

### Step 2: N-cyclopropyl-3-fluoro-4-methyl-5-[3-[[1-[2-[2-(methylamino)ethoxy]phenyl]cyclopropyl]amino]-2-oxo-pyrazin-1-yl]benzamide

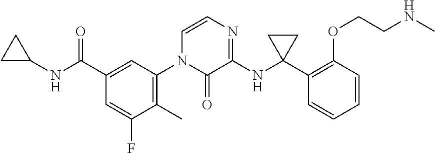 [SMILES: CNCCOc1ccccc1C1(Nc2nccn(-c3cc(C(=O)NC4CC4)cc(F)c3C)c2=O)CC1]

A suspension of benzyl-[2-[2-[1-[[4-[5-(cyclopropylcarbamoyl)-3-fluoro-2-methyl-phenyl]-3-oxo-pyrazin-2-yl]amino]cyclopropyl]phenoxy]ethyl]-methyl-ammonium hemi-oxalate (10.29 g, 15.9 mmol, 1.0 eqv) and Pd—C(Johnson Mattey type 5R87 L wet paste, 3% w/w Pd, 425 mg, 0.12 mmol, 0.0075 eqv) in a mixture of 1:1 iso-propanol:water (100 ml, 10 rel vol) was hydrogenated at 50° C. and 4 bar hydrogen pressure for 4 h. At the end of this period the pH of the mixture was adjusted to 12.5-13.0 using 25% w/w aqueous sodium hydroxide solution (approximately 1.9 mL) at 20° C. The mixture was then heated to 30° C. and filtered through a Harborlite® bed to remove the palladium on carbon. The filter cake was washed with iso-propanol (20 ml, 2 rel vol) and the filtrate was combined with the main batch. Iso-propyl acetate (80 mL, 8 rel vol) was added to the combined filtrates to give a two phase mixture which was then separated and the lower aqueous phase was re-extracted with more isopropyl acetate (80 mL, 8 rel vol). The aqueous phase was then discarded and the combined organic phases were washed with 25% w/w aqueous NaCl solution (50 mL, 5 rel vol) and then water (50 mL, 5 rel vol). The organic phase was then distilled at 300 mbar with the bath temperature set at 65° C.-70° C. (vapour temperature: approx. 50° C.) until 70 mL (7 rel vol) of solution remained. More iso-propyl acetate (100 mL, 10 rel vol) was added to the mixture and the distillation was continued at 300 mbar until 60 mL (6 rel vol) of the mixture remained. At this point the moisture content of the mixture was measured, to ensure it was less than <0.5% w/w [if it is higher than this, then more iso-propyl acetate (5 rel vol) should be added and the distillation repeated until the correct moisture level is obtained]. The temperature of the mixture was adjusted to 60° C. and the batch was seeded with N-cyclopropyl-3-fluoro-4-methyl-5-[3-[[1-[2-[2-(methylamino)ethoxy]phenyl]cyclopropyl]-amino]-2-oxo-pyrazin-1-yl]benzamide (10 mg, 0.1% w/w based on input starting material). [Seeds were obtained by retrieving an aliquot of the hot solution and allowing this to cool to ambient temperature until spontaneous crystallisation occurred]. Distillation was continued until 40 mL (4 rel vol) of the mixture remained. The temperature of the batch was not allowed to fall below 60° C. n-Heptane (40 mL, 4 rel vol) was added dropwise to the mixture at 60° C. over 1 hour and then the suspension was cooled to 20° C. over 3 h and stirred at 20° C. for 4 h. The product was then filtered and pulled dry and then washed with n-heptane (20 mL, 2 rel vol) and pulled dry. The product was then dried in a vacuum oven at 50° C. to give N-cyclopropyl-3-fluoro-4-methyl-5-[3-[[1-[2-[2-(methylamino)ethoxy]phenyl]cyclopropyl]-amino]-2-oxo-pyrazin-1-yl]benzamide (7.4 g, 90% yield) as a free flowing solid.

¹H-NMR (δ, CDCl₃, 400 MHz): 0.51-0.53 (m, 2H), 0.65-0.70 (m, 2H), 1.02-1.05 (m, 1H), 1.18 (s, 3H), 1.96 (s, 3H), 2.38 (s, 3H), 2.82-2.90 (m, 3H), 4.05 (t, 5.52 Hz, 2H), 6.74 (d, 4.52 Hz, 1H), 6.83-6.88 (m, 2H), 6.96 (d, 8.04 Hz, 1H), 7.17-7.21 (m, 1H), 7.48-7.51 (m, 2H), 7.60 (s, 1H), 7.73 (d, 10.00 Hz, 1H), 8.46 (d, 4.00 Hz, 1H).

¹³C-NMR (δ, CDCl₃, 100.6 MHz): 5.6, 9.9, 13.8, 14.0, 23.1, 32.1, 36.1, 50.4, 67.4, 111.9, 114.1, 114.4, 117.3, 119.5, 121.7, 121.9, 125.5, 125.7, 128.3, 129.7, 130.3, 133.9, 134.0, 139.9, 150.1, 151.0, 157.8, 159.1, 161.5, 164.7, 164.7.

### Step 3: Recrystallisation to give N-cyclopropyl-3-fluoro-4-methyl-5-[3-[[1-[2-[2-(methylamino)ethoxy]phenyl]cyclopropyl]amino]-2-oxo-pyrazin-1-yl]benzamide, Form S

Crude N-cyclopropyl-3-fluoro-4-methyl-5-[3-[[1-[2-[2-(methylamino)ethoxy]phenyl]cyclopropyl]amino]-2-oxo-pyrazin-1-yl]benzamide (20.0 g, 407 mmol, 1.0 eqv) was charged to a vessel followed by acetonitrile (170 mL, 8.5 rel vol). The mixture was heated to reflux for 1 h and then screened into a second vessel to remove extraneous matter. The first vessel was rinsed with acetonitrile (10 mL, 0.5 rel vol), which was also screened into the second vessel. The acetonitrile solution was heated to 70° C. for 15 minutes before being cooled to 55° C. A suspension of seed N-cyclopropyl-3-fluoro-4-methyl-5-[3-[[1-[2-[2-(methylamino)ethoxy]phenyl]cyclopropyl]amino]-2-oxo-pyrazin-1-yl]benzamide Form S (140 mg) in acetonitrile (1 mL, 0.05 rel vol) was charged to the clear solution and the resulting suspension was held at 55° C. for 10 h. [Seeds of Form S were obtained by retrieving an aliquot of the hot solution and allowing this to cool slowly to ambient temperature]. The resulting slurry was then cooled to 5° C. over a period of 20 h. The suspension was filtered and the filter cake was washed twice with MTBE (each wash 40 mL, 2 rel vol). After drying in a vacuum oven at 50° C. for 20 h N-cyclopropyl-3-fluoro-4-methyl-5-[3-[[1-[2-[2-(methylamino)ethoxy]phenyl]cyclopropyl]amino]-2-oxo-pyrazin-1-yl]benzamide Form S (16.6 g, 83% yield) was isolated as a granular solid.

¹H-NMR (δ, CDCl₃, 400 MHz): 0.51-0.53 (m, 2H), 0.65-0.70 (m, 2H), 1.02-1.05 (m, 1H), 1.18 (s, 3H), 1.96 (s, 3H), 2.38 (s, 3H), 2.82-2.90 (m, 3H), 4.05 (t, 5.52 Hz, 2H), 6.74 (d, 4.52 Hz, 1H), 6.83-6.88 (m, 2H), 6.96 (d, 8.04 Hz, 1H), 7.17-7.21 (m, 1H), 7.48-7.51 (m, 2H), 7.60 (s, 1H), 7.73 (d, 10.00 Hz, 1H), 8.46 (d, 4.00 Hz, 1H).

¹³C-NMR (δ, CDCl₃, 100.6 MHz): 5.6, 9.9, 13.8, 14.0, 23.1, 32.1, 36.1, 50.4, 67.4, 111.9, 114.1, 114.4, 117.3, 119.5, 121.7, 121.9, 125.5, 125.7, 128.3, 129.7, 130.3, 133.9, 134.0, 139.9, 150.1, 151.0, 157.8, 159.1, 161.5, 164.7, 164.7.

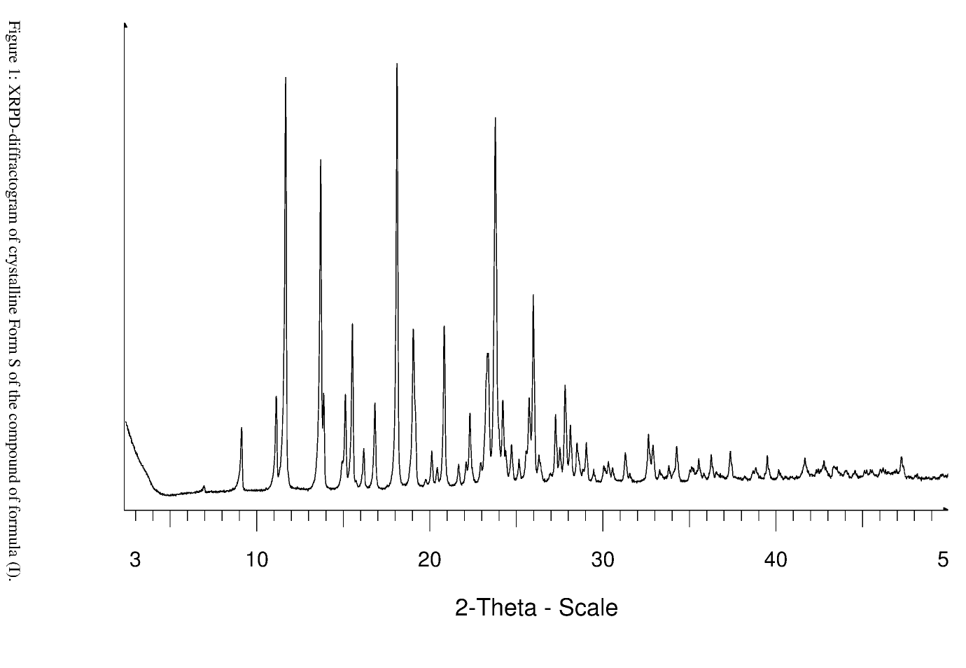

The XRPD diffractogram of Form S of the compound of formula (I) obtained by way of Example 5, step 3 is shown in FIG. 1 below.

X-Ray powder diffraction peaks are shown in Table A.

TABLE A
X-Ray Powder Diffraction peaks for N-
cyclopropyl-3-fluoro-4-methyl-5-[3-[[1-[2-[2-
(methylamino)ethoxy]phenyl]cyclopropyl]amino]-2-
oxo-pyrazin-1-yl]benzamide Form S

| Angle 2-Theta (2θ) | Intensity |
|---|---|
| [CuK 1.5418 Å radiation] | % |
| 9.1 | 16 |
| 11.6 | 97 |
| 13.7 | 77 |
| 15.1 | 22 |
| 15.5 | 39 |
| 16.2 | 9 |
| 16.8 | 21 |
| 18.1 | 100 |
| 20.8 | 38 |
| 23.8 | 86 |

### Example 6: Comparison of the Chemical Stability of Form S of the Compound of Formula (I) with Form A

Crystalline particles of Form S of the compound of formula (I) were obtained using the methods described in Example 5 above. Crystalline particles of Form A were also obtained, using the ethyl acetate-heptane crystallization system described in WO2010/071583. Samples of Form A and Form S were subject to accelerated degradation studies. Samples of each crystalline form were stored at varying combinations of temperature and relative humidity and analysed for purity by HPLC at 0, 14 and 28 days. The results of the study are shown in Table B. It can be seen that, under all the conditions that the samples were exposed to, Form S showed significantly less degradation than Form A.

TABLE B
Effect of accelerated degradation on the purity of
samples of N-cyclopropyl-3-fluoro-4-methyl-5-[3-[[1-[2-[2-
(methylamino)ethoxy]phenyl]cyclopropyl]amino]-2-
oxo-pyrazin-1-yl]benzamide Form A & Form S

| | | Relative | Purity by HPLC/% | | |
|---|---|---|---|---|---|
| Form | Temp/° C. | Humidity/% | Day 0 | Day 14 | Day 28 |
| A | 60 | 30 | 99.43 | 99.41 | 99.39 |
| | 60 | 75 | 99.43 | 99.20 | 99.21 |
| | 70 | 11 | 99.43 | 99.33 | 99.33 |
| | 70 | 75 | 99.43 | 98.78 | 98.76 |
| | 80 | 30 | 99.43 | 99.40 | 99.36 |
| S | 60 | 30 | 99.77 | 99.77 | 99.77 |
| | 60 | 75 | 99.77 | 99.64 | 99.63 |
| | 70 | 11 | 99.77 | 99.77 | 99.77 |
| | 70 | 75 | 99.77 | 99.77 | 99.77 |
| | 80 | 30 | 99.77 | 99.77 | 99.78 |

The above description of illustrative embodiments is intended only to acquaint others skilled in the art with Applicant's specification, its principles, and its practical application so that others skilled in the art may readily adapt and apply the specification in its numerous forms, as they may be best suited to the requirements of a particular use. This description and its specific examples, while indicating embodiments of this application, are intended for purposes of illustration only. This specification, therefore, is not limited to the illustrative embodiments described in this specification, and may be variously modified. In addition, it is to be appreciated that various features of the specification that are, for clarity reasons, described in the context of separate embodiments, also may be combined to form a single embodiment. Conversely, various features of the specification that are, for brevity reasons, described in the context of a single embodiment, also may be combined to form sub-combinations thereof.

1. A crystalline form of the compound of formula (I): 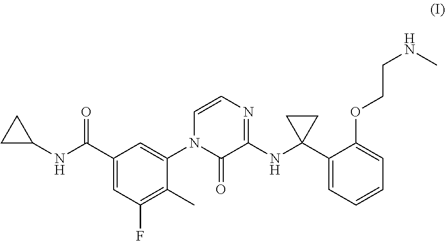 [SMILES: CNCCOc1ccccc1C1(Nc2nccn(-c3cc(C(=O)NC4CC4)cc(F)c3C)c2=O)CC1]
which has an X-ray powder diffraction pattern with at least one specific peak selected from the peaks at about 2-theta=9.1, 15.1, 16.2, 16.8 and 23.8°.

2. A crystalline form of the compound of formula (I), as claimed in claim 1, which has an X-ray powder diffraction pattern with at least two specific peaks at about 2-theta=9.1° and 15.1°.

3. A crystalline form of the compound of formula (I), as claimed in claim 1, which has an X-ray powder diffraction pattern with specific peaks at about 2-theta=9.1, 15.1, 16.2, 16.8 and 23.8°.

4. A crystalline form of the compound of formula (I), as claimed in claim 1, which has an X-ray powder diffraction pattern with specific peaks at about 2-theta=9.1, 11.6, 13.7, 15.1, 15.5, 16.2, 16.8, 18.1, 20.8 and 23.8°.

5. A crystalline form of the compound of formula (I), as claimed in claim 1, which has an X-ray powder diffraction pattern substantially the same as the X-ray powder diffraction pattern shown in FIG. 1.

6. A process for the production of a crystalline form of the compound of formula (I), as claimed in claims 1 to 5, comprising crystallizing the compound from a solution, suspension or slurry of the compound of formula (I) in a solvent system containing acetonitrile.

7. A pharmaceutical composition that comprises a crystalline form of the compound of formula (I) as claimed in claims 1 to 5, and a pharmaceutically acceptable diluent or carrier.

8. (canceled)

9. (canceled)

10. A method of treating chronic obstructive pulmonary disease in a warm-blooded animal, which comprises administering to a mammal in need of such treatment a therapeutically effective amount of a crystalline form of the compound of formula (I), as claimed in claims 1 to 5.

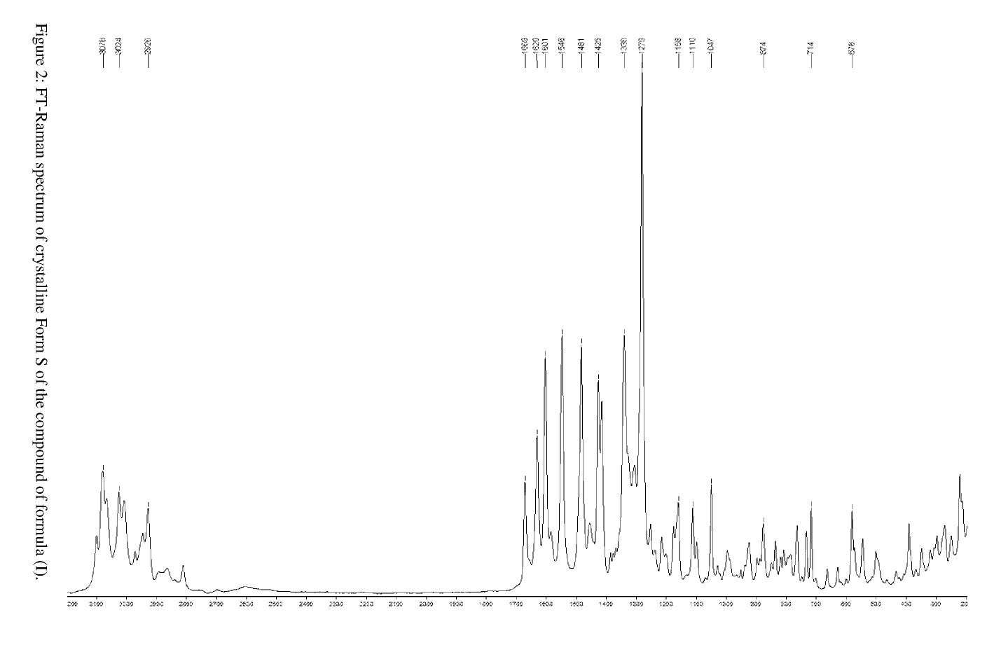

11. A crystalline form of the compound of formula (I), as claimed in claim 1, which has an FT-Raman spectrum substantially the same as the FT-Raman spectrum shown in FIG. 2.

12. A method of treating asthma in a warm-blooded animal, which comprises administering to a mammal in need of such treatment a therapeutically effective amount of a crystalline form of the compound of formula (I), as claimed in claims 1 to 5.
